(19) United States
(12) Patent Application Publication
(10) Pub. No.: US 2025/0359982 A1
(43) Pub. Date: Nov. 27, 2025

(54) AORTIC ROOT IMPLANTS AND RELATED SYSTEMS AND METHODS

(71) Applicant: inQBB Medical Technologies, LLC, Burlington, MA (US)

(72) Inventors: Arshad Quadri, West Hartford, CT (US); J. Brent Ratz, Burlington, MA (US); Garrett Johnson, Costa Mesa, CA (US); Levi John Nicholas Caffes, Somerville, MA (US); Caroline Elizabeth Buckley, Belmont, MA (US); Jack Berkman Sattell, Cambridge, MA (US)

(73) Assignee: inQBB Medical Technologies, LLC, Burlington, MA (US)

(21) Appl. No.: 19/170,241

(22) Filed: Apr. 4, 2025

Related U.S. Application Data
(60) Provisional application No. 63/575,153, filed on Apr. 5, 2024.
(60) Provisional application No. 63/575,287, filed on Apr. 5, 2024.
(60) Provisional application No. 63/712,328, filed on Oct. 25, 2024.
(60) Provisional application No. 63/712,368, filed on Oct. 25, 2024.

(51) Int. Cl. A61F 2/07; A61F 2/06; A61F 2/24

(52) U.S. Cl. CPC A61F 2/07; A61F 2/24; A61F 2002/061; A61F 2250/0007; A61F 2250/0039; A61F 2250/0069

(57) ABSTRACT

The present disclosure generally relates to implantable medical devices, and, in some embodiments, to a prosthetic implant. Such implantable devices may be, in some cases, useful in the treatment of acute aortic dissections (AADs). In some embodiments, the prosthetic implant comprises an inner frame and an outer frame, the inner frame comprising an expandable support structure. The expandable support structure may comprise one or more expandable branches. The expandable branch may comprise a proximal portion, a middle portion, and a distal portion configured to permit flattening, telescoping, pivoting, and/or expansion of the one or more portions of the expandable branch. The one or more expandable branches may be, in some cases, configured to be placed into a vessel, e.g., a coronary artery. Additionally, a first expandable support structure comprising one or more expandable branches may be, in some cases, coupled to a second expandable support structure comprising one or more expandable branches.

### RELATED APPLICATIONS

This Application is a Non-Provisional of Provisional (35 USC 119(e)) of U.S. Application Ser. No. 63/712,328, filed Oct. 25, 2024, entitled “AORTIC ROOT IMPLANTS AND RELATED SYSTEMS AND METHOD” and a Non-Provisional of Provisional (35 USC 119(e)) of U.S. Application Ser. No. 63/712,368, filed Oct. 25, 2024, entitled “ROOT GRAFT IMPLANTS AND RELATED METHODS” and a Non-Provisional of Provisional (35 USC 119(e)) of U.S. Application Ser. No. 63/575,153, filed Apr. 5, 2024, entitled “AORTIC ROOT IMPLANTS AND RELATED SYSTEMS AND METHOD” and a Non-Provisional of Provisional (35 USC 119(e)) of U.S. Application Ser. No. 63/575,287, filed Apr. 5, 2024, entitled “ROOT GRAFT IMPLANTS AND RELATED METHODS”. The entire contents of these applications are incorporated herein by reference in their entirety.

### FIELD

The present invention generally relates to implantable medical devices and, more particularly, to prosthetic aortic implants, as well as systems and methods involving the same. Such devices, systems, and methods may be, in some cases, useful for e.g., the treatment of Acute Aortic Dissections (AAD), Intramural Hematomas and Thoracic Aortic Aneurysms.

### BACKGROUND

Management of AADs depend on the type of dissection and its location along the aorta, but generally involves medications, to reduce heart rate and lower blood pressure which help to prevent the ADD from worsening, and/or surgery, to remove as much of the dissected aorta as possible and to stop blood from leaking into the aortic wall. However, nearly 10-30% of all AADs are deemed inoperable and managed primarily with medication alone. The mortality in this population is high, with approximately 15-30% of patients dying within 24 hrs, which tapers off to approximately 1% per day from day 6 through day 30. Outcomes for surgical candidates are equally poor with sequela rates, e.g., mortality and neurological damage, as high as 15-30%. Accordingly, improved devices and methods are needed.

### SUMMARY

The present invention generally relates to implantable medical devices, and, more particularly, to a prosthetic aortic implant, systems comprising the prosthetic aortic implant, and related methods. The subject matter of the present disclosure involves, in some cases, interrelated products, alternative solutions to a particular problem, and/or a plurality of different uses of one or more systems and/or articles.

Aspects of the present disclosure generally relate to a prosthetic implant configured to be positioned within an aortic root of a native aorta. In some embodiments, the implant comprises an expandable root support structure comprising one or more openings and one or more expandable branches configured to be positioned within the one or more openings. In some embodiments, the expandable root support structure is configured to be positioned within at least a portion of an ascending portion of a native aorta. In some embodiments, the expandable root support structure comprises a valved conduit positioned at a proximal end of the expandable root support structure, and a non-porous layer that is configured to contact an outer wall of the native aorta. In some embodiments, the one or more expandable branches comprises a telescoping structure comprising a proximal portion, middle portion, and a distal portion, wherein an inner diameter of the proximal portion is larger than an inner diameter of the distal portion. In some embodiments, the one or more expandable branches is configured to be positioned within at least a portion of at least one coronary artery of an aortic root and to permit blood flow from within the expandable root support structure and into at least one coronary artery of the aortic root.

Some aspects generally relate to other prosthetic implants, e.g., for use in other medical indications. For example, in some embodiments, the implant comprises an expandable support structure comprising one or more openings and one or more expandable branches configured to be positioned within the one or more openings. In some embodiments, the one or more expandable branches comprise a telescoping structure comprising a proximal portion, a middle portion, and a distal portion. In some embodiments, the proximal portion is configured to pivot the one or more expandable branches between 0 degrees and 360 degrees, relative to a first axis.

Some aspects generally relate to a prosthetic implant configured to be positioned within an aortic root and an aortic arch of a native aorta. In some embodiments, the implant comprises an expandable root support structure, comprising one or more openings and one or more expandable branches configured to be positioned within the one or more openings, sized and configured to be positioned within an ascending portion of a native aorta. In some embodiments, the implant further comprises an expandable arch support structure, comprising one or more openings and one or more expandable branches configured to be positioned within the one or more openings, sized and configured to be positioned within a descending portion of a native aorta. In some embodiments, the expandable root support structure comprises a first non-porous layer and is configured to contact an outer wall of the native aorta, and one or more expandable branches comprising a telescoping structure and/or a tapered geometry. In some embodiments, the one or more expandable branches is configured to be positioned within at least a portion of at least one coronary artery of an aortic root of the native aorta, wherein upon implantation in a subject, permits blood flow from within the expandable root support structure into at least one coronary artery of the aortic root of the native aorta. In some embodiments, the proximal end of the expandable root support structure comprises a valved conduit. In some embodiments, a distal end of the expandable root support structure is configured to engage a proximal end of the expandable arch support structure.

Some aspects of the present disclosure generally relate to a dual frame prosthetic implant. In some embodiments, the prosthetic implant comprises an inner frame operably linked to an outer frame, the inner frame comprising an expandable root support structure comprising one or more openings and one or more expandable branches configured to be positioned within the one or more openings, and operably linked to the expandable root support structure. In some embodiments, the expandable root support structure is configured to be positioned within at least a portion of an ascending portion of a native aorta. In some embodiments, the expandable root support structure comprises a valved conduit positioned at a proximal end of the expandable root support structure, and a non-porous layer that is configured to contact an outer wall of the native aorta. In some embodiments, the one or more expandable branches comprises a telescoping structure comprising a proximal portion and a distal portion, wherein an inner diameter of the proximal portion is larger than an inner diameter of the distal portion. In some embodiments, the one or more expandable branches is configured to be positioned within at least a portion of at least one coronary artery of an aortic root and to permit blood flow from within the expandable root support structure and into at least one coronary artery of the aortic root.

Some aspects of the present disclosure generally relate to methods. In some embodiments, the methods are directed to a method of treating an aortic dissection. In some embodiments, the methods comprise advancing a first guide wire into an ascending aorta. In some embodiments, the methods comprise advancing a prosthetic implant delivery system into the ascending aorta over the first guidewire, the prosthetic implant delivery system comprising an outer sheath and a distal sheath extending through the outer sheath, the distal sheath carrying a distal end of an expandable root support structure. In some embodiments, the methods comprise retracting the outer sheath in the ascending aorta to expose the distal sheath over the first guidewire. In some embodiments, the methods comprise advancing the distal sheath over the first guidewire to expose at least part of the distal portion of the expandable root support structure. In some embodiments, the methods comprise retracting the outer sheath out of the ascending aorta to expose a proximal portion of the expandable root support structure, wherein the proximal portion comprises an aortic valve. In some embodiments, the methods comprise advancing a first expandable branch of the expandable root support structure into a coronary artery over a first coronary access wire, wherein the first expandable branch comprises a telescoping structure comprising a proximal portion, a middle portion, and a distal portion, and wherein the first expandable branch is configured to pivot the between 0 degrees and 360 degrees, relative to a first axis. In some embodiments, the methods comprise advancing the distal sheath to fully expose the distal portion of the expandable root support structure from the distal sheath.

In some embodiments, the methods are directed to methods of treating a dissection. In some embodiments, the methods comprise advancing a first guide wire into a descending aorta into an ascending aorta. In some embodiments, the methods comprise advancing a prosthetic implant delivery system through the descending aorta and into the ascending aorta over the first guidewire, the prosthetic implant delivery system comprising an outer sheath and a proximal sheath extending through the outer sheath, the outer sheath carrying an expandable root support structure. In some embodiments, the methods comprise retracting the outer sheath toward the descending aorta to expose the proximal sheath over the first guidewire. In some embodiments, the methods comprise advancing the proximal sheath out of the ascending aorta to expose a proximal portion of the expandable root support structure, wherein the proximal portion comprises an aortic valve. In some embodiments, the methods comprise further retracting the outer sheath toward the descending aorta to expose at least a portion of a distal portion of the expandable support structure. In some embodiments, the methods comprise advancing a first expandable branch of the expandable support structure into a coronary artery over a first coronary access wire, wherein the first expandable branch comprises a telescoping structure comprising a proximal portion, a middle portion, and a distal portion, and wherein the first expandable branch is configured to pivot the expandable branch between 0 degrees and 360 degrees, relative to a first axis. In some embodiments, the methods comprise retracting the outer sheath into the descending aorta to fully expose distal portion of the expandable root support structure within the ascending aorta.

Some aspects of the disclosure generally relate to a graft delivery system. In some embodiments, the graft delivery system comprises a guide wire lumen, a distal capsule comprising a plurality of bearing elements, a distal locking ring and a proximal locking ring, and a prosthetic implant system comprising an expandable root support structure. In some embodiments, a proximal end of the expandable root support structure is connected to the proximal locking ring. In some embodiments, a distal end of the expandable root support structure is connected to the distal locking ring. In some embodiments, the plurality of bearing elements, the distal locking ring, and the proximal locking ring are configured to rotate about a guide wire lumen.

In some embodiments, the graft delivery system comprises an outer sheath, the outer sheath configured to move along a first guidewire. In some embodiments, the graft delivery system comprises a distal sheath extending through the outer sheath, the outer sheath carrying an expandable root support structure. In some embodiments, the graft delivery system comprises a first expandable branch of the expandable support structure inside or extending from the outer sheath, the first expandable branch configured to move along a first coronary access wire.

In another aspect, the present disclosure generally encompasses methods of using one or more of the embodiments described herein, for example, administration of a prosthetic aortic implant. In still another aspect, the present disclosure encompasses methods of using one or more of the embodiments described herein, for example, prosthetic aortic implant.

Other advantages and novel features of the present invention will become apparent from the following detailed description of various non-limiting embodiments of the invention when considered in conjunction with the accompanying figures. In cases where the present specification and a document incorporated by reference include conflicting and/or inconsistent disclosure, the present specification shall control.

### BRIEF DESCRIPTION OF DRAWINGS

Non-limiting embodiments of the present disclosure will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the disclosure shown where illustration is not necessary to allow those of ordinary skill in the art to understand the disclosure. In the figures:

FIG. 1 shows an exemplary expandable root support structure, according to one set of embodiments. In some embodiments, the exemplary root support structure comprises a coupling structure at an ascending portion (e.g., proximal portion) of the support structure for coupling to an expandable arch support structure. In some embodiments, the exemplary expandable root support structure comprises one or more openings configured to receive one or more expandable branches configured for placement into either a right and/or left coronary artery.

FIG. 2A shows an exemplary expandable root support structure comprising a coupling structure at a proximal end for coupling to a distal end of an expandable root support structure and one or more expandable branches operably linked to said support structure and configured for placement into either a right and/or left coronary artery, according to one set of embodiments.

FIGS. 2B and 2C show exemplary embodiments of an expandable branch of an expandable root support structure, according to one set of embodiments. In some embodiments, the exemplary expandable branch comprises a proximal portion, a middle portion, and a distal portion, wherein the distal portion is positioned within at least a portion of the middle portion. In some embodiments, the proximal portion comprises a nonporous layer such as ePTFE and does not comprise a metal frame. In some embodiments, the middle portion comprises a metallic wire frame (e.g., a telescoping frame) comprising a nonporous layer (e.g., ePTFE). In some embodiments, the middle portion comprises a tapered end comprising one or more pores in the nonporous layer. In some embodiments, the distal portion comprises an expandable support structure (e.g., a distal expandable frame) comprising a nonporous layer (e.g., ePTFE covering). In some embodiments, when the distal portion is in a crimped state, the one or more pores are open, and when in an expanded state (FIG. 2B), the one or more pores are closed (FIG. 2C).

FIG. 2D shows an exemplary embodiment of an expandable branch operably linked to an expandable root support structure, according to one set of embodiments. In some embodiments, the expandable branch is operably linked to the expandable root support structure using sutures. In some embodiments, the expandable branch comprises a middle portion comprising a metallic wire frame (e.g., telescoping frame) that permits extension and inversion of the branch. In some embodiments, the expandable branch comprises a distal portion comprising an expandable support structure (e.g., a distal expandable frame) that permits crimping and expansion of a distal end, for example, within a coronary vessel.

FIG. 3A shows a side view of an exemplary aortic root implant, according to one set of embodiments. The aortic root implant comprises an expandable root support structure comprises a coupling structure at a proximal end for coupling to a distal end of an expandable root support structure, one or more expandable branches operably linked to said support structure and configured for placement into either a right and/or left coronary artery, and a valved conduit at a proximal end of the expandable root support structure.

FIG. 3B shows an interior lumen of an exemplary aortic root implant, according to one set of embodiments. In some embodiments, the aortic root implant comprises an expandable root support structure, one or more openings configured to receive on or more expandable branches, and a valved conduit comprising a trilobed valve.

FIG. 4A shows a side view and FIG. 4B shows an interior view, respectively, of an exemplary aortic arch implant, according to one set of embodiments. In some embodiments, the aortic arch implant comprises an expandable arch support structure comprising one or more openings configured to receive one or more expandable branches configured for placement into one or more head vessels of an aortic arch.

FIG. 4C shows a side view of an exemplary expandable branch configured to be placed within one or more holes of an expandable arch support structure, according to one set of embodiments. As shown in the figure, the expandable branches comprise a proximal portion, middle portion, and distal portion. In some embodiments, the proximal portion comprises a nonporous layer comprising a tubular shape and the distal portion comprises a distal expandable frame. In some embodiments, the middle portion comprises at least a portion of the distal expandable frame and the nonporous layer. In some embodiments, the middle and distal portions are configured to expand within at least a portion of at least one head vessel of an aortic arch.

FIG. 5 shows an exemplary embodiment of a modular implant, according to one set of embodiments. In some embodiments, the modular implant comprises an expandable root support structure of an aortic root implant coupled to an expandable arch support structure of an aortic arch implant. In some embodiments, the modular implant is configured to be positioned within at least a portion of the descending aorta, the aortic arch, the ascending aorta and into the aortic root. In some embodiments, the one or more expandable branches of the expandable root support structure are configured to be placed in a left coronary artery and/or a right coronary artery. In some embodiments, one or more expandable branches of the expandable arch support structure are configured to be placed in a brachiocephalic artery, a left common carotid artery, and/or a subclavian artery.

FIG. 6 shows an exemplary aortic root implant partially loaded onto an exemplary delivery system, according to one set of embodiments. The telescoping structure of the one or more expandable branches allows the branches to be reversibly inverted into, or extended from, the body of the aortic root implant.

FIG. 7 shows an exemplary aortic arch implant loaded onto an exemplary delivery system, according to one set of embodiments. The telescoping structure of the one or more expandable branches allows the branches to be reversibly inverted into, or extended from, the body of the aortic arch implant. Additionally, or alternatively, the telescoping structure also permits folding of the one or more branches into a configuration that is parallel to the body of the aortic arch implant (e.g., expandable arch support structure).

FIG. 8 illustrates the anatomic anchoring and/or sealing zones of a modular implant, according to one set of embodiments. The anchoring and/or sealing zones of the implant serve to anchor the implant to the native aorta while preventing blood from leaking out of the implant.

FIG. 9A illustrates the flexibility of an expandable branch, according to one set of embodiments. In some embodiments, the one or more expandable branches is configured to pivot radially between 0 degrees and 360 degrees, relative to a first axis. In some embodiments, the one or more expandable branches is configured to pivot laterally between 90 degrees and −90 degrees, relative to a second axis. In some embodiments, the first axis runs parallel to an elongate central passageway defined by the one or more expandable branches (e.g., a lumen). In some embodiments, the second axis runs perpendicular to the elongate central passageway defined by the one or more expandable branches (e.g., the second axis is parallel to the expandable root/arch support structure). FIG. 9B illustrates the positioning of a first expandable branch relative to a second expandable branch on an exemplary aortic root implant. In some embodiments, the branches are separated by about 120 degrees, and/or generally corresponds to the anatomical positioning of the right and left coronary ostia in the aortic root.

FIG. 10 illustrates an exemplary dual framed aortic root implant comprising an inner frame and an outer frame, according to one set of embodiments.

FIGS. 11A and 11B illustrates anchor points between an inner frame and an outer frame of a dual framed aortic root implant. In some embodiments, the outer frames comprise a plurality of anchor points, for example, to anchor the outer frame to the inner frame. In some embodiments, the inner frame comprises a plurality of anchor points. In some embodiments, both the inner frame and outer frame comprise a plurality of anchor points.

FIGS. 12A-12C illustrate additional exemplary embodiments of an outer frame of a dual framed aortic root implant, as contemplated herein.

FIGS. 13A-13E illustrate various embodiments of an exemplary dual framed aortic root implant further comprising a plurality of leaflet anchors. FIG. 13A shows an outer frame of the dual framed aortic root implant, the outer frame comprising a plurality of leaflet anchors. FIG. 13B shows an inner frame anchored to an outer frame comprising the plurality of leaflet anchors. FIG. 13C illustrates the relative position of the leaflet anchors to the native annulus of a native heart valve. FIG. 13D illustrates the placement of a dual framed implant along the native annular plane. FIG. 13E illustrates an outer frame comprising a plurality of leaflet anchors with the leaflet anchor crimped in an upright position.

FIGS. 14A and 14B shows an exemplary embodiment of a prosthetic implant delivery system as contemplated herein. As shown in FIG. 1B, the exemplary system comprises a nosecone over a first guidewire and a distal sheath covering a distal portion of an expandable root support structure (e.g., covered graft). The delivery system further comprises a guide catheter (FIG. 14A), a balloon catheter (FIG. 14A), and an outer sheath (FIG. 14B).

FIGS. 15A-15I illustrate an exemplary set of embodiments wherein a prosthetic implant delivery system is delivered transapically. A guide wire is first passed into the ascending aorta, and the delivery system is tracked up the guidewire (guidewire not shown in FIG. 15A). In some embodiments, the outer sheath is retracted to a proximal edge of a coronary ostium to expose a distal sheath (FIG. 15B). In some embodiments, the distal sheath is pushed distally to deploy a distal portion of the graft, and the outer sheath is further retracted to deploy a valve at a proximal end of an expandable root support structure (FIG. 15C). In some embodiments, a guide catheter is articulated to point a distal tip of a balloon catheter toward a coronary artery. The guide catheter is continually articulated and pushed into the coronary artery until access is gained (FIG. 15D). Once coronary access is established, blood may be perfused through the delivery system and through the coronary arteries to maintain blood flow to the heart (FIG. 15E). In some embodiments, a balloon catheter, located within a lumen of the guide catheter, is advances an expandable branch of the expandable root support structure into the coronary artery. In some embodiments the expandable branch comprises a chameleon eye structure which permits the branch to be articulated and extended. In some embodiments, a distal end of the expandable branch comprises is expanded using the balloon catheter, thus anchoring the implant to the native aorta (FIG. 15F). In some embodiments, the ballon catheter is retracted, and the process is repeated to in order to place a second expandable branch of the expandable root support structure into the other coronary artery (FIGS. 15G-15H). Lastly, the distal sheath is pushed distally to deploy a distal portion of the expandable root support structure (FIG. 15I).

FIGS. 16A-16H illustrate an exemplary set of embodiments wherein a prosthetic implant delivery system is delivered transfemorally, according to some embodiments.

FIG. 17 illustrates the maneuverability of a coronary accesses wire and/or coronary access catheter. As shown in the figure, the coronary access wire or catheter can rotate about and move along an axis that is parallel to one or more openings in the expandable root support structure.

FIGS. 18A and 18B illustrate an exemplary prosthetic implant delivery system configured to permit independent rotation of an exemplary prosthetic implant loaded within the delivery system (from top to bottom).

FIG. 19 is a computer aided drawing illustrating the assembly of the nosecone and the distal locking ring.

FIG. 20 illustrates the connection between the distal locking ring and the distal end of an exemplary implant.

FIGS. 21A and 21B illustrates transfemoral delivery of a prosthetic implant using the prosthetic implant delivery system disclosed herein. The FIG. 21A shows a coronary access catheter in a first position. FIG. 21B shows that the coronary access catheter has been rotated by 180 degrees relative to the first position. Rotation is accomplished via a knob on the handle of the delivery device (see FIG. 18A for exemplary location of rotation knob on device).

### DETAILED DESCRIPTION

The detailed description set forth below describes various configurations of the subject technology and is not intended to represent the only configurations in which the subject technology may be practiced. The detailed description includes specific details for the purpose of providing a thorough understanding of the subject technology. Accordingly, dimensions may be provided in regard to certain aspects as non-limiting examples. However, it will be apparent to those skilled in the art that the subject technology may be practiced without these specific details. In some instances, well-known structures and components are shown in block diagram form in order to avoid obscuring the concepts of the subject technology.

The present disclosure generally relates to implantable medical devices, and, in some embodiments, to a prosthetic implant. Such implantable devices may be, in some cases, useful in the treatment of acute aortic dissections (AADs). In some embodiments, the prosthetic implant comprises an expandable support structure. The expandable support structure may comprise one or more expandable branches. The expandable branch may comprise a proximal portion, a middle portion, and a distal portion configured to permit flattening, telescoping, pivoting, and/or expansion of the one or more portions of the expandable branch. The one or more expandable branches may be, in some cases, configured to be placed into a vessel, e.g., coronary vessel or aortic arch head vessel. Additionally, a first expandable support structure comprising one or more expandable branches may be, in some cases, coupled to a second expandable support structure comprising one or more expandable branches.

In some embodiments, the prosthetic implants is implanted (e.g., surgically) in a subject to treat a disease, disorder, or other clinically recognized condition, or for prophylactic purposes, and/or may have a clinically significant effect on the body of the subject to treat and/or prevent the disease, disorder, or condition. A “subject” refers to any animal such as a mammal (e.g., a human). Non-limiting examples of suitable subjects include a human, a non-human primate, a cow, a horse, a pig, a sheep, a goat, a dog, a cat or a rodent such as a mouse, a rat, a hamster, a bird, a fish, or a guinea pig. Generally, the invention is directed toward use with humans. In some embodiments, a subject may demonstrate health benefits, e.g., upon implantation of the prosthetic aortic implant.

In some embodiments, the prosthetic devices disclosed herein are useful for the treatment of subjects suffering from one or more types of Acute Aortic Dissections (AADs). As would be understood by those of ordinary skill in the art, AADs generally occur when a portion of the aortic intima (the inner most layer of the aorta) ruptures and systemic blood pressure serves to delaminate the intimal layer from the media layer resulting in a false lumen for blood flow that can propagate in multiple directions along the length of the aorta. AADs that occur in the ascending portion of the aorta may generally be classified as Acute Type A Aortic Dissections (ATAADs, also referred to as Type 1 and Type 2 according to De Bakey classification system), whereas those not involving the ascending aorta are referred to as Type B dissections (according to the Stanford classification system). In some cases, failure to rapidly treat AADs, and particularly, ATAADs, may lead to severe sequela including stroke, organ damage, e.g., kidney failure or life-threatening intestinal damage, aortic valve damage, and death due to severe internal bleed (e.g., mortality rate is nearly 50% at 48 hours post injury and 90% within 30 days post injury).

Those of ordinary skill will understand, based upon the teachings of this specification that the systems, methods, and devices described herein may, in some embodiments, fill an important therapeutic gap in the treatment of patients with AADs. For example, the prosthetic aortic implants described herein may advantageously be useful for providing a prophylactic that may be, in some cases, administered non-invasively in an outpatient setting. In some embodiments, the prosthetic aortic implants described herein may advantageously be administered to patients with recently diagnosed aortic aneurysms, for example, as a preventative measure to delay (or prevent) disease progression. In some embodiments, the prosthetic aortic implants may advantageously be useful for rapidly treating patients suffering from ATAADs (e.g., an aortic dissection in the ascending aorta that occur acutely and rapidly without warning, as may occur in patients with undiagnosed aortic aneurysms). In some embodiments, placement (e.g., implantation) of the prosthetic aortic implant within the ascending aorta of a patient suffering from ATAAD may serve to reinforce the inner wall of the aorta near the dissection and re-establish a true lumen for blood to flow through. In some embodiments, the prosthetic aortic implants described herein may advantageously provide a non-invasive method to fix damaged aortic valves, for example, by incorporating a valve frame configured to reversibly (or irreversibly) receive a prosthetic aortic valve. For example, in some embodiments, the prosthetic aortic implants described herein is sized and configured to receive (e.g., reversibly) a transcatheter aortic valve implant (TAVI). In some embodiments, an aortic valve such as a TAVI is positioned within the prosthetic aortic implant and/or a portion of the native aorta that has been configured to receive the TAVI as a result of the presence of the prosthetic aortic implant.

The prosthetic aortic implants described herein may have several advantages over previously described devices. For example, some previously described devices generally comprise a short one-piece implant constructed of fabric with built-in reinforcements configured to reside within the ascending aorta alone. However, such devices may be, in some cases, prone to movement and dislocation e.g., because they generally lack features that may anchor the device to the native aorta. In contrast to traditional devices, the prosthetic implants described herein comprise, in some embodiments, one or more expandable anchoring structures, configured to engage and apply a radial outward force, to one or more structures of a native aorta, e.g., aortic sinuses and/or the sinotubular junction, within an aortic root of the native aorta, thus anchoring the device to the native aorta (e.g., reducing the likelihood of movement and/or dislocation). In some embodiments, the disclosed devices are configured to extend from the ascending aorta into the descending aorta, wherein the descending portion further anchors the device to the native aorta, e.g., advantageously further reducing the likelihood of movement and/or dislocation. Additionally, in some embodiments, the prosthetic implants described herein comprise one or more expandable branches, configured to engage and apply a radial outward force, to one or more structures of a native aorta, e.g., a right and/or left coronary artery.

In some embodiments, aortic grafts for treating aortic aneurysms are used to treat ATAADs, wherein the aortic grafts generally comprise a non-porous layer to wall off the aneurysm from the main lumen of the graft and aorta. However, such grafts cannot generally be placed over regions of the aorta (e.g., the aortic arch) e.g., that require fenestration windows so blood may flow to branched vessels. The prosthetic implants described herein may advantageously comprise, in some embodiments, one or more expandable branches configured to sit within a branched vessel (e.g., brachiocephalic artery, left common carotid artery, left subclavian artery, left and/or right coronary arteries). Additionally, in alternative embodiments, the prosthetic implants described herein may comprise a porous layer positioned over at least part of an expandable support structure, e.g., thereby permitting the graft to span from the ascending aorta into the descending aorta without blocking blood flow to critical branch vessels (e.g., brachiocephalic artery, left common carotid artery, and the left subclavian artery).

In some cases, bare-metal implants have also been described for the treatment of AADs. However, bare-metal frames are generally abrasive and may erode through the tissue and/or cause the fragile intima layer to dissect further. The prosthetic implants described herein may advantageously comprise, in some embodiments, an expandable support structure comprising an atraumatic nonporous outer layer configured to distribute a radial force throughout the entire aorta, e.g., thereby reducing the likelihood of the aneurysms rupturing.

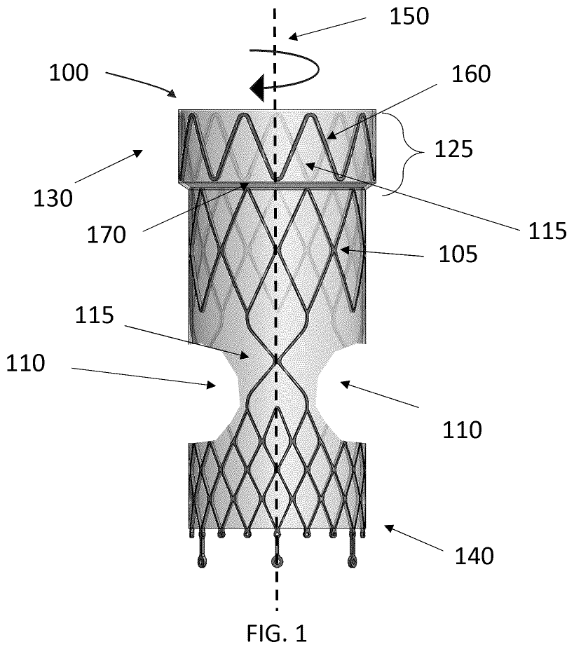

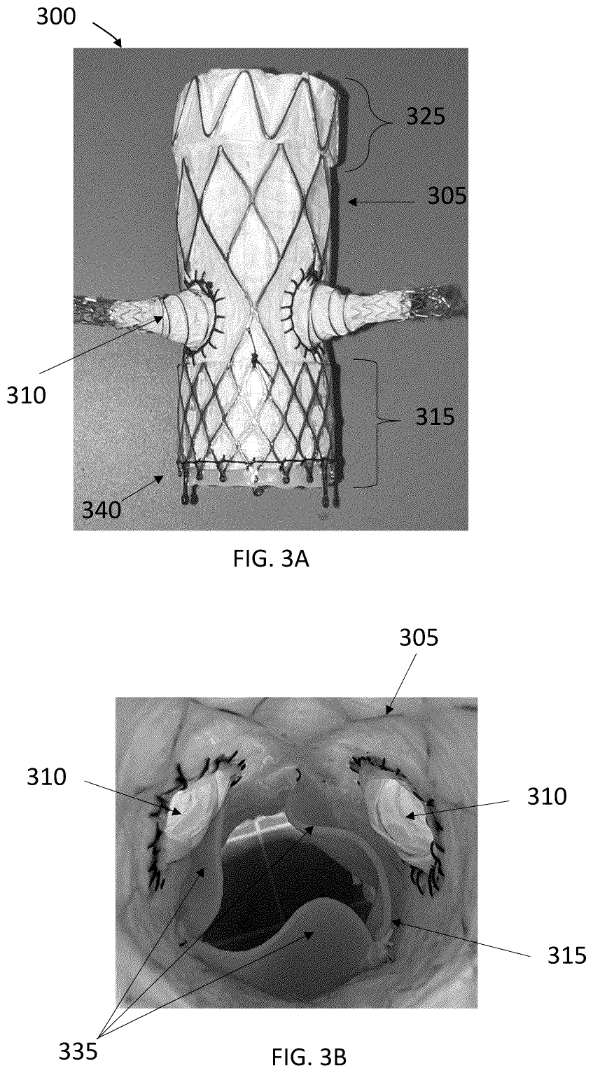

The prosthetic implants of the present disclosure are generally directed toward aortic root implants, aortic arch implants, and modular implants comprising an aortic root implant coupled to an aortic arch implant. In some embodiments, an aortic root implant is configured to be placed within an aortic root of a subject. FIG. 1A shows a side view of an exemplary aortic root implant comprising an expandable root support structure 100 configured to be positioned within an aortic root of a subject. In some embodiments, expandable root support structure 100 comprising frame 105. In some embodiments, frame 105 is tubular comprising a central lumen (see FIG. 3B) that extends from a proximal end 140 to a distal end 130 of expandable support structure 100.

In some embodiments, expandable root support structure 100 further comprises nonporous layer 115, which may be provided over, within, or interwoven into frame 105 of expandable root support structure 100. Nonporous layer 115 comprises, according to some embodiments, a fabric or polymer. Exemplary embodiments, include but are not limited to, one or a combination of polyester, nylon, polytetrafluoroethylene (PTFE), expanded PTFE (ePTFE), or silicone.

In some embodiments, expandable root support structure 100 further comprises coupling structure 125 at distal end 130 of expandable root support structure 100. Those of skill in the art will understand that coupling structure 125 is intended to couple expandable root support structure 100 to a second prosthetic implant (e.g., an aortic arch implant configured to sit within an aortic arch of a subject). In some embodiments, coupling structure 125 comprises frame 160, which may be part of, or separate from, frame 105. In some embodiments, frame 160 is tubular comprising a central lumen that aligns with the central lumen of frame 106. Frame 160, according to some embodiments, may be a wire frame or wire coil with a zigzag or Z-shaped pattern along a cylindrical portion of the coil. Other patterns suitable for coupling the aortic root implant to a second prosthetic implant are also possible in some embodiments. For example, in some embodiments, frame 160 has a braided configuration, a sine wave pattern, or a trilobe pattern. In some embodiments, frame 160 is a coiled wire forming a wire frame, such as, for example, a coiled ribbon. In some embodiments, frame 160 may be formed from one or more of a metal (e.g., stainless steel, nitinol, or the like), a polymer, a biological material, a bio-absorbable material, and/or other suitable material.

In some embodiments, coupling structure 125 further comprises nonporous layer 115, which may be provided over, within, or interwoven into frame 160 of coupling structure 125. As described elsewhere herein, nonporous layer 115 comprises, according to some embodiments, a fabric or polymer. Exemplary embodiments, include but are not limited to, one or a combination of polyester, nylon, polytetrafluoroethylene (PTFE), expanded PTFE (ePTFE), or silicone.

In some embodiments, lip 170, as shown in FIG. 1, is formed at the interface of coupling structure 125 with frame 105 of expandable root support structure 100. Those of skill in the art will understand that lip 170, is useful, according to some embodiments, for engaging and securing expandable root support structure 100 to a second prosthetic implant (e.g., an aortic arch implant as discussed elsewhere herein).

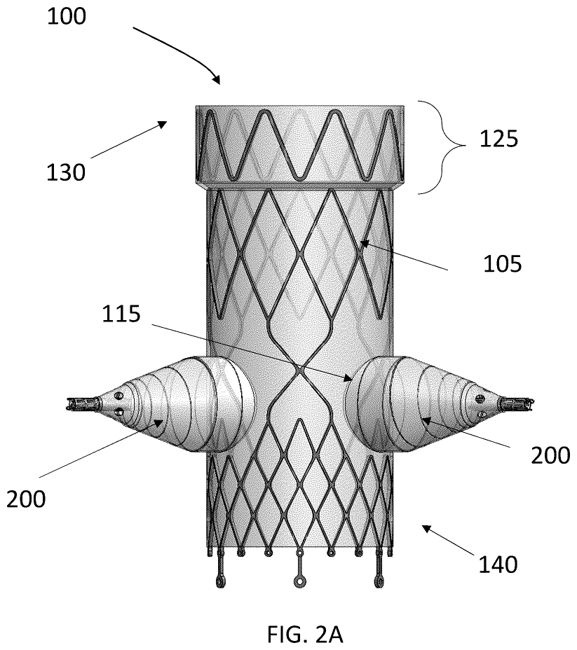

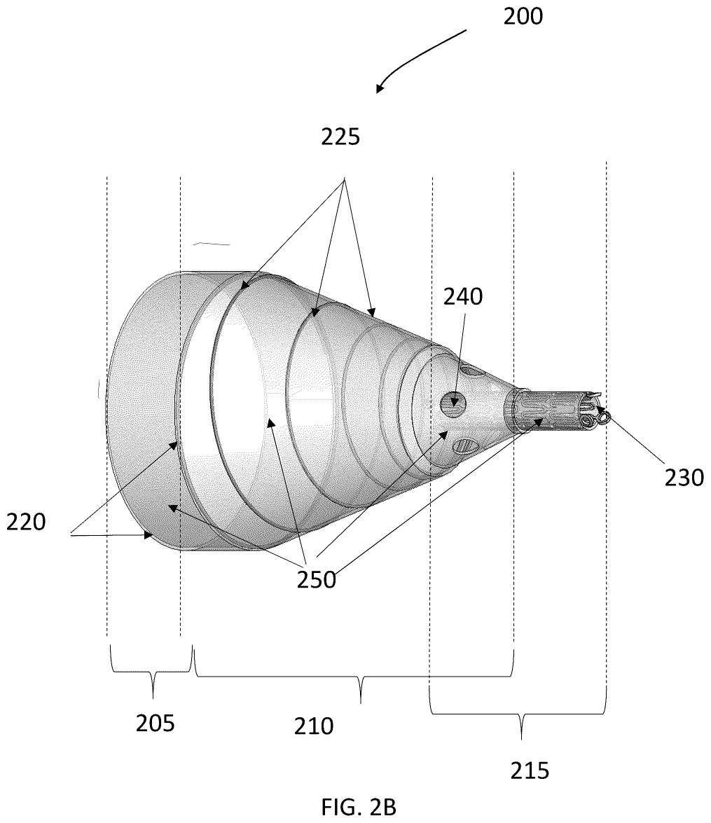

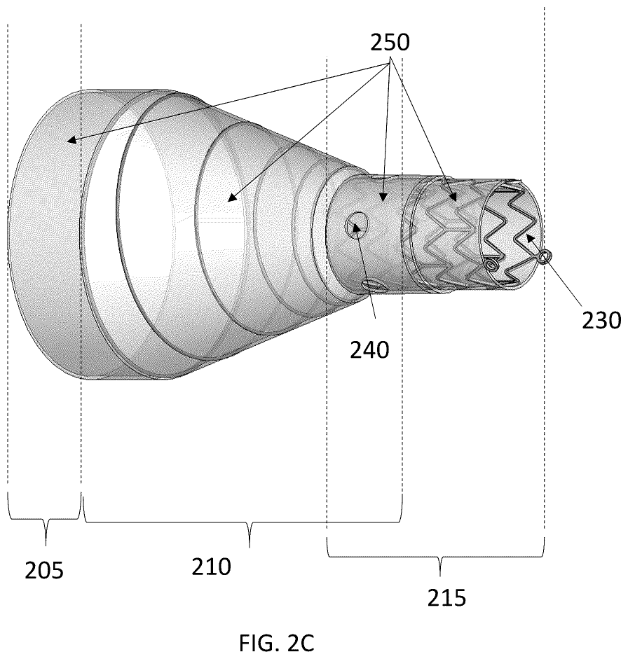

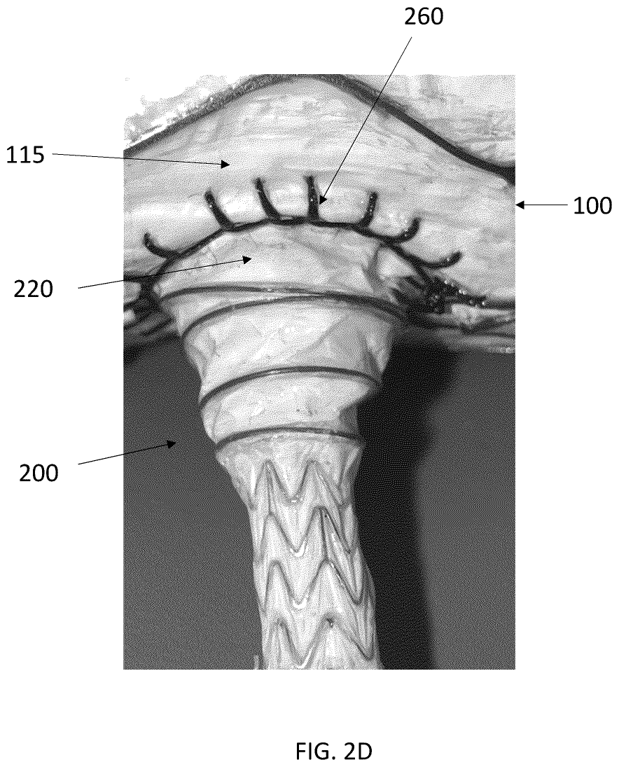

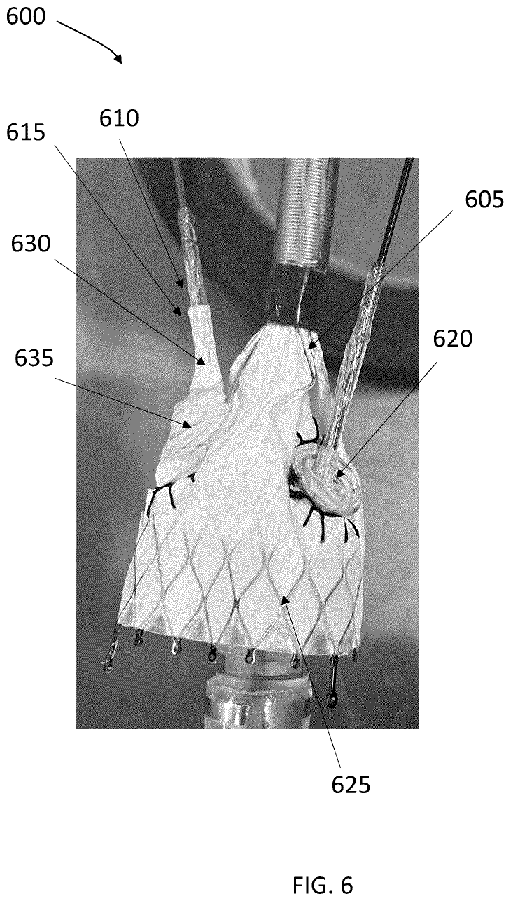

FIG. 2 shows exemplary expandable root support structure 100 comprising one or more expandable branches 200 configured to be positioned within one or more openings 110. In some embodiments, one or more branches 200 comprise a telescoping structure comprising proximal portion 205, middle portion 210, and distal portion 215, as shown in FIG. 2B. Those of skill in the art will appreciate and understand that the telescoping structure of the one or more expandable branches enables the expandable branch to be configured in an extended state (e.g., distal to expandable root support structure as shown in FIG. 6) or in a collapsed state (e.g., proximal to expandable root support structure and/or at least partially within a lumen of expandable root support structure, as shown in FIG. 6).

In some embodiments, proximal portion 205 comprises telescoping frame 220. In some embodiments, middle portion 210 comprises telescoping frame 225. In some embodiments, telescoping frame 225 comprises a tapered geometry. In some embodiments, telescoping frames 220 and 225 are formed from a single wire frame. In some embodiments, telescoping frames 220 and 225 are formed from separate wire frames. In some embodiments, distal portion 215 comprises distal expandable frame 230. In some embodiments, distal expandable frame 230 is positioned at least partially within telescoping frame 225 (e.g., the distal portion is positioned at least partially within the middle portion). In some embodiments, distal expandable frame 230 is positioned at least partially within, and at a tapered end, of telescoping frame 225.

Telescoping frames 220 and 225 of the proximal and middle portions, respectively, and distal expandable frame 230 may be, in some cases, formed from one or more of a metal (e.g., stainless steel, nitinol, or the like), a polymer, a biological material, a bio-absorbable material, and/or other suitable material. Likewise, in some embodiments, telescoping frames 220 and 225 of the proximal portion 205 and middle portion 210, respectively, and distal expandable frame 230 of distal portion 215 may comprise any suitable structure and/or geometry known to the skilled artisan. For example, in some embodiments, telescoping frames 220 and 225 and/or distal expandable frame 230 is a wire frame or wire coil. In some embodiments, the wire coil comprises a tapered geometry. In some embodiments, telescoping frames 220 and 225, and/or distal expandable frame 235 may comprise a wire frame or wire coil with a zigzag or Z-shaped pattern along a cylindrical portion of the coil. Other suitable structures and/or geometries may include, for example, braided configurations, a sine wave pattern, or a trilobe pattern. In some embodiments still, telescoping frames 220 and 225 and/or distal expandable frame 230 comprise a coiled wire forming a wire frame, such as, for example, a coiled ribbon.

In some embodiments, one or more expandable branches 200 further comprises nonporous layer 250. Nonporous layer 250 comprises, according to some embodiments, a fabric or polymer, such as for example, one or a combination of polyester, nylon, polytetrafluoroethylene (PTFE), expanded PTFE (ePTFE), or silicone. In some embodiments, nonporous layer 250 is ePTFE.

In some embodiments, nonporous layer 250 is provided over, within, or interwoven into telescoping frames 220 and 225 and/or distal expandable frame 230. In some embodiments, nonporous layer 250 comprises one or more pores at the tapered end of telescoping frame 225.

In some embodiments, one or more pores 240 in nonporous layer 250 are open when distal expandable frame 230 of distal portion 215 is in a crimped configuration, as shown in FIG. 2B. Such configurations are useful, for example, for preserving coronary perfusion during placement of the prosthetic implant within the coronary ostia. In some embodiments, the one or more of pores 240 are configured to be in a closed position when distal expandable frame 230 is in an expanded position, as shown in FIG. 2C. The skill artisan will understand that upon expansion, at least a portion of distal expandable frame 230 comprising nonporous layer 250 radially compresses against the tapered end of telescoping frame 225, thus sealing the one or more pores 240 at the tapered end of telescoping frame 225. Such configurations may be useful, in some cases, for example, for preventing leakage of body fluid from the expandable branch after placement at a target location (e.g., coronary vessels).

In some embodiments, one or more expandable branches is configured to sit within one or more openings, and is adjacent (e.g., operably linked to), an expandable root support structure. The one or more expandable branches may be, in some cases, secured within the one or more openings of the expandable root support structure using any suitable technique known to those of skill in the art. For example, as shown in FIG. 2D, in some embodiments, the one or more expandable branches is secured within the one or more openings of the expandable root support structure using suture 260 (e.g., proximal end 220 is sutured to nonporous layer 115 using suture 260). Any suitable suture material and/or suture knot known to the skilled artisan may be, in some cases, used to secure the expandable branch within the one or more openings. However, the skilled artisan will understand and appreciate that other methods (e.g., without sutures) of attaching the one or more expandable branches to the one or more openings of the expandable root support structure. For example, in some embodiments, the one or more expandable branches is secured to the one or more openings of the expandable root support structure using a nonporous layer. In some embodiments, the nonporous layer is ePTFE.

As used herein, when a component is referred to as being “adjacent” another component, it can be directly adjacent to (e.g., in contact with) the component, or one or more intervening components also may be present. A component that is “directly adjacent” another component means that no intervening component(s) is present. For example, two or more components may be adjacent (e.g., operably) linked such that the two components are directly operably linked such that no intervening component(s) are present, or there may be one or more intervening linking features between the two components.

In some embodiments, a prosthetic implant, as disclosed herein, comprises a valved conduit. FIG. 3A shows a side view of an exemplary prosthetic implant 300 comprising an expandable root support structure 305, one or more expandable branches 310, coupling structure 325, and valved conduit 315 at proximal end 140. FIG. 3B shows an interior view of exemplary prosthetic implant 300 having valved conduit 315 comprising tricuspid valve 235 at proximal end 340 of said implant. In some embodiments, the one or more expandable branches 310 are proximal to valved conduit 315. In some embodiments, coupling structure 325 is distal to valved conduit 315. FIG. 3B also shows valved conduit 315 comprising tricuspid valve 335 (e.g., tri-leaflet). The skilled artisan will understand and appreciate, however, that in some embodiments the valved conduit may comprise any suitable valve design known in the art, such as for example, a bicuspid valve design or a modular design that enables reversible attachment of the valve to the expandable root support structure.

In some embodiments, valved conduit 315 comprises an aortic valve frame. In some embodiments, the aortic frame may be configured to receive an aortic valve implant, either during (or after) deployment of the device. In some embodiments, a proximal end of the prosthetic aortic valve frame is flared and extends into a left ventricular outflow tract (LVOT). In some embodiments, placing the flared proximal end within the LVOT anchors the device to the native aorta, thus reducing the likelihood of undesired movement and dislocation.

In some embodiments, an aortic valve frame comprises a “bridge” valve, for example, to serve as a temporary valve (e.g., <24 hrs). In some embodiments, a permanent valve, e.g., a commercially available TAVR (transcatheter aortic valve replacement), may be placed within the bridge valve between about 24 hours to 48 hours post deployment of the prosthetic aortic implant, for example, using a non-invasive percutaneous approach. In some embodiments, the aortic valve frame may comprise a destination valve, for example, as a permanent aortic valve replacement option. In some embodiments, the aortic valve frame may be configured to reversibly (or irreversibly) receive the aortic valve implant. The ability to repeatedly remove the valve, for example, during percutaneous placement of a TAVR may permit optimal fitting of the prosthetic within the bridge valve. In some embodiments, the aortic valve may comprise a tri-lobe (or tri-leaflet) design (e.g. to mimic the native aortic valve) and may comprise a bioprosthetic material (e.g., porcine or bovine aortic valves) or a synthetic material (e.g., Dacron or the like).

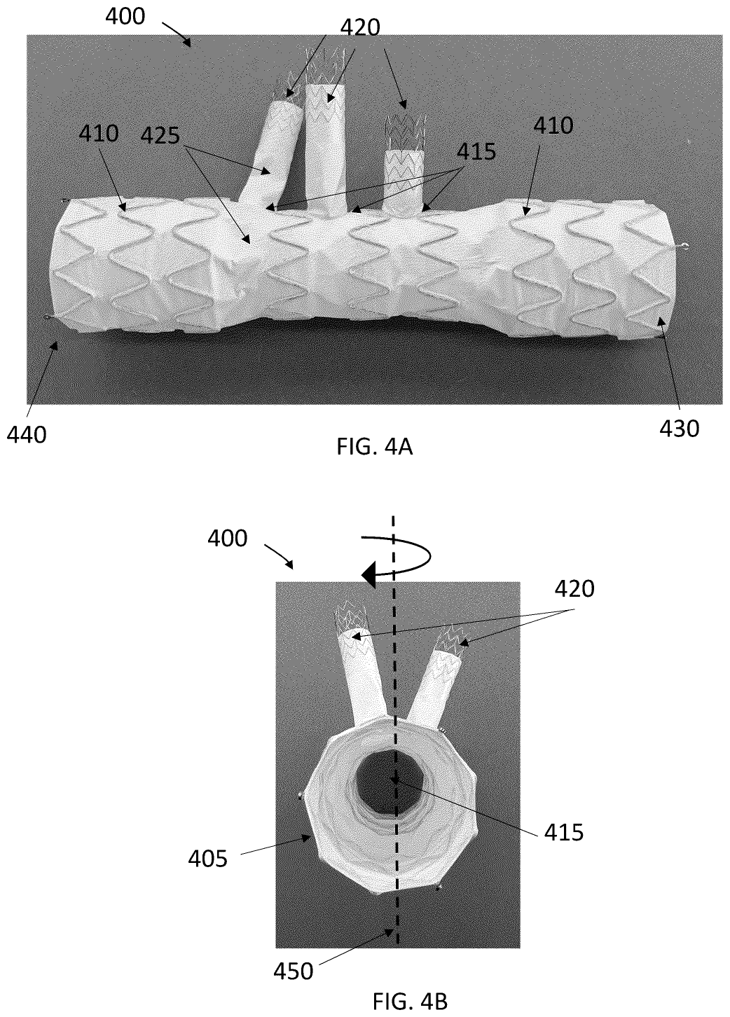

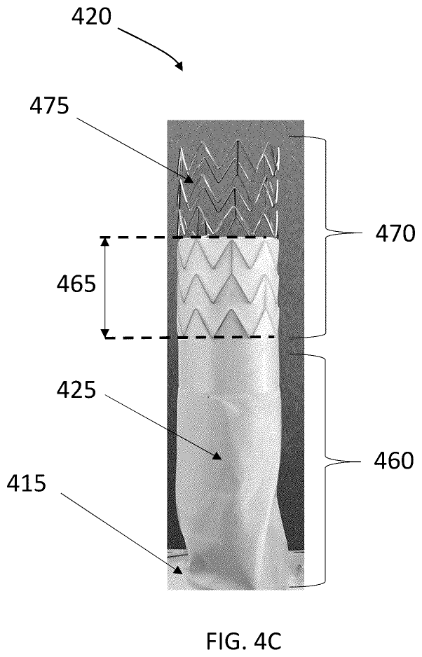

Other aspects of the disclosure generally relate to a prosthetic implant configured to be positioned within an aortic arch of a subject and/or to engage a second prosthetic implant positioned within an aortic root of said subject. FIGS. 4A and 4B show a side view and front view of exemplary prosthetic implant 400, respectively. In some embodiments, exemplary prosthetic implant 400 comprises expandable arch support structure 405 comprising frame 410. In some embodiments, frame 410 is tubular comprising central lumen 415 that extends from a proximal end 440 to a distal end 430 of expandable arch support structure 405. Frame 410, according to some embodiments, may be a wire frame or wire coil with a zigzag or Z-shaped pattern along a cylindrical portion of the coil. The skilled artisan will understand, however, that frame 410 may comprise other patterns suitable for treating an aortic dissection. For example, in some embodiments, frame 410 may have a braided configuration, a sine wave pattern, a trilobe pattern. In some embodiments, frame 410 is a coiled wire forming a wire frame, such as, for example, a coiled ribbon. In some embodiments, frame 410 may be formed from one or more of a metal (e.g., stainless steel, nitinol, or the like), a polymer, a biological material, a bio-absorbable material, and/or other suitable material.

In some embodiments, frame 410 may be constructed using a combination of structures and/or geometries (e.g., braided and zig-zag patterns). For example, in some embodiments, the frame has a distal end 430 having a braided configuration and a proximal end 440 having a wire frame with a zig-zag pattern along the cylindrical portion of the coil. In some embodiments, the frame comprises a middle portion (e.g., in between the distal and proximal ends) having a third structure/geometry that is the same, or different, than the proximal and/or distal ends. In some embodiments, the distal end, middle portion, and proximal end of the frame have the same structure/geometry. In some embodiments, the frame has a braided configuration.

In some embodiments, expandable arch support structure 405 further comprises one or more openings 415 configured to receive one or more expandable branches 420 comprising a telescoping structure.

In some embodiments, expandable arch support structure 405 further comprises nonporous layer 425, which may be provided over, within, or interwoven into frame 410 of expandable arch support structure 405. Nonporous layer 425 comprises, according to some embodiments, a fabric or polymer, such as for example, one or a combination of polyester, nylon, polytetrafluoroethylene (PTFE), expanded PTFE (ePTFE), or silicone. Other materials are also possible.

As mentioned above, FIGS. 4A and 4B illustrate, according to some embodiments, one or more expandable branches 420 comprising a telescoping structure configured to be positioned within one or more openings 415 of expandable arch support structure 405. In some embodiments, one or more expandable branches 420 is tubular comprising a central lumen that extends from a proximal portion 460 to a distal portion 470 of the one or more expandable branches 420. In some embodiments, the central lumen of the one or more expandable branches is in fluidic communication with the central lumen of the expandable arch support structure.

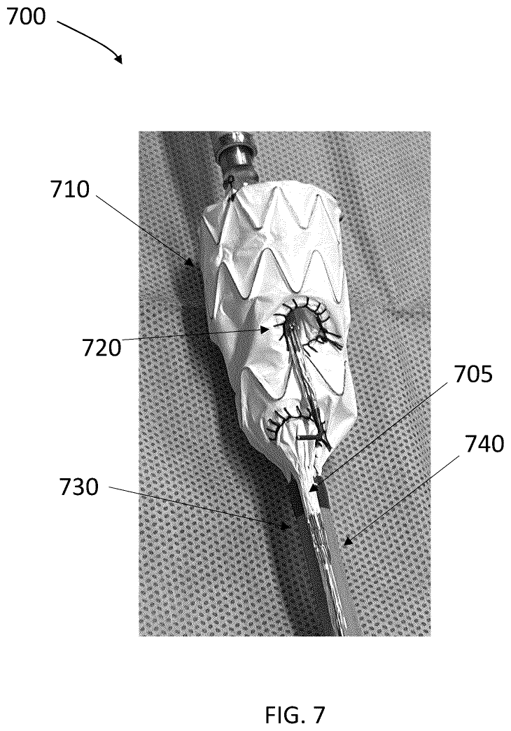

FIG. 4C illustrates a telescoping structure of the one or more expandable branches 420, according to some embodiments. In some embodiments, the one or more branches 420 comprises proximal portion 460, middle portion 465, and distal portion 470. As shown in FIG. 4C, in some embodiments, proximal portion 460 comprises a telescoping frame comprising nonporous layer 425 having a tubular and/or cylindrical structure. However, this embodiment is not limiting, and nonporous layer 425 may be, in some cases, configured into any suitable geometry known to the skilled artisan for use as contemplated herein. In some embodiments, proximal portion 460 does not contain a metal frame therewith (e.g., an expandable frame is not present with nonporous layer 425). In some embodiments, such configurations are useful, for example, for folding the expandable branch into a configuration parallel to the expandable arch support structure and/or inverting the expandable branch into the lumen of the expandable arch support structure (e.g., for delivery and/or deployment of said implant as shown in FIG. 7). Those of skill in the art will understand, however, that the figures are not meant to be limiting in any way, and that proximal portion 460 may comprise a metallic frame (e.g., a telescoping metal frame or expandable frame) in some embodiments. In some embodiments, proximal portion 460 may comprise a frame (e.g., metallic frame) and nonporous layer 425.

In some embodiments, one or more branches 420 comprises a distal portion 470 comprising a distal expandable frame 475. In some embodiments, distal expandable frame 475 is configured to expand from a crimped/closed position into an expanded position within at least one of the head vessels of the aortic arch (e.g., brachiocephalic artery, left subclavian artery, and/or the left common carotid artery). In some embodiments, expansion from a crimped/closed position into an expanded position anchors the implant to the head vessel.

In some embodiments, middle portion 465 comprises a portion of distal expandable frame 475 and nonporous layer 425. In some embodiments, nonporous layer 425 is provided over, within, or interwoven into distal expandable frame 475 within middle portion 465 of the one or more expandable branches. Those of skill in the art will understand and appreciate that upon expansion, at least a portion of distal expandable frame 475, middle portion 465 radially compresses against the intima of the head vessel (e.g., pressing nonporous layer 425 against interior wall of head vessel). In some embodiments, such configurations are useful, for example, for preventing leakage of body fluid (e.g., blood) from the lumen of expandable branch after placement at a target location (e.g., brachiocephalic artery, left subclavian artery, and/or the left common carotid artery).

In some embodiments, one or more expandable branches are operably linked to an expandable arch support structure. The one or more expandable branches may be, in some cases, secured within the one or more openings of the expandable arch support structure using any suitable technique known to those of skill in the art. For example, in some embodiments, the one or more expandable branches is secured within the one or more openings of the expandable arch support structure using a suture (e.g., the nonporous layer of the proximal end is sutured to the nonporous layer of the expandable arch support structure using suture material). In some embodiments, any suitable suture material and/or suture knot known to the skilled artisan may be used to secure the expandable branch within the one or more openings of the expandable arch support structure. However, the skilled artisan will understand and appreciate that other methods (e.g., without sutures) of attaching the one or more expandable branches to the one or more openings of the expandable root support structure. For example, in some embodiments, the one or more expandable branches is secured to the one or more openings of the expandable root support structure using a nonporous layer. In some embodiments, the nonporous layer is ePTFE.

The one or more telescoping frames and/or expandable frames of the proximal, middle, and/or distal portions of the one or more expandable branches positioned within the one or more openings of an expandable arch support structure may be, in some cases, formed from one or more of a metal (e.g., stainless steel, nitinol, or the like), a polymer, a biological material, a bio-absorbable material, and/or other suitable material. Likewise, in some embodiments, the telescoping frames and/or expandable frames of the proximal, middle, and/or distal portion of the one or more expandable branches may comprise any suitable structure and/or geometry known to the skilled artisan. For example, in some embodiments, said frames comprise a wire frame or wire coil. In some embodiments, the wire coil comprises a tapered geometry. In some embodiments, the frames may comprise a wire frame or wire coil with a zigzag or Z-shaped pattern along a cylindrical portion of the coil. Other suitable structures and/or geometries may include, for example, braided configurations, a sine wave pattern, or a trilobe pattern. In some embodiments, the frames comprise a coiled wire forming a wire frame, such as, for example, a coiled ribbon.

Additional aspects of the disclosure generally relate to a modular prosthetic implant comprising an aortic root implant coupled to an aortic arch implant. In some embodiments, the aortic root implant and/or aortic arch implant of the modular prosthetic implant is any aortic root implant and/or aortic arch implant disclosed herein. In some embodiments, the modular prosthetic implant is configured to extend from the descending aorta to the ascending aorta and curve along with the curvature of the aortic arch when expanded within the aorta.

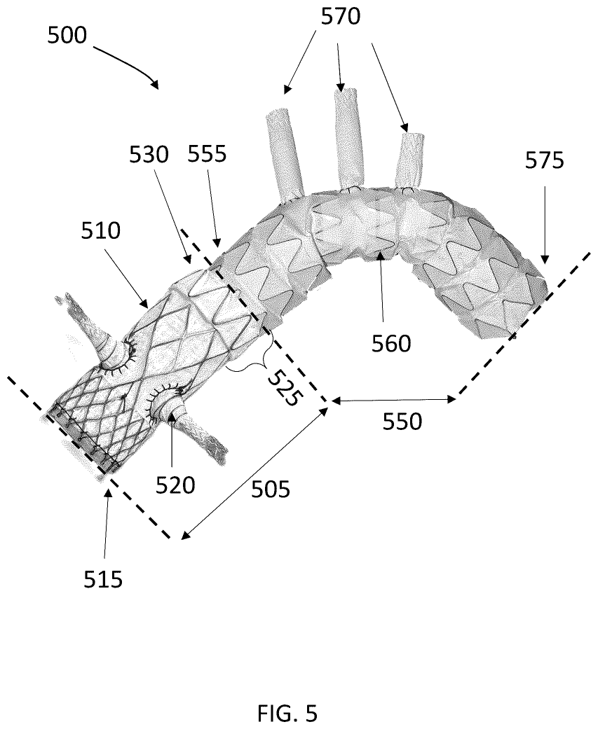

FIG. 5 shows exemplary modular prosthetic implant 500 as contemplated herein. In some embodiments, a distal end 530 of aortic root implant 505 is coupled to a proximal end 555 of aortic arch implant 550 via coupling structure 525. As described elsewhere herein, aortic root implant 505, comprises expandable root support structure 510 and one or more expandable branches 520, which is configured to sit within an aortic root and the coronary ostia, respectively, of a subject. In some embodiments, the one or more expandable branches 520, upon expansion within the coronary ostia, is configured to apply a radial force to the coronary arteries, thus anchoring said implant to the aortic root. Additionally, in some embodiments, expandable root support structure may further comprise an expandable anchoring structure at proximal end 515. In some embodiments, the expandable anchoring structure may be configured to be positioned within the aortic root of a subject and apply radial force to one or more of the sinuses of the aortic root and/or the sinotubular junction when expanded.

In some embodiments, aortic arch implant 550 comprises expandable arch support structure 560 configured to extend from the descending aorta, through the aortic arch and into the ascending aorta of a subject. In some embodiments, aortic arch implant 550 comprises one or more expandable branches 570 configured to sit within at least one head vessel of the aortic arch (e.g., brachiocephalic artery, left subclavian artery, and/or left common carotid artery). In some embodiments, the one or more expandable branches 570, upon expansion within the head vessels, is configured to apply a radial force to the intima of the head vessels, thus anchoring said implant to the aortic arch of a native aorta. Additionally, a distal end 575 of expandable arch support structure 560, upon expansion within at least a portion of the descending aorta is configured to apply a radial force to the intima of the descending aorta further anchoring the implant to the native aorta.

FIG. 6 illustrates an exemplary aortic root implant 605 partially loaded within exemplary aortic root delivery system 600. In some embodiments, one or more expandable branches 610 of exemplary aortic root implant 605 comprises a telescoping structure. For example, in some embodiments one or more expandable branches 610 are configured to be in an expanded state 615 or a collapsed state 620 (e.g., inverted such that at least a portion of the branch is collapsed within a lumen of expandable root support structure 625). In some cases, a distal portion 630 and/or a middle portion 635 are at least partially collapsed into a lumen (e.g., collapsed state 620) of the expandable root support structure. Again, it is believed that such configurations are advantageous, for example, for delivering the implant to the desired anatomical location.

FIG. 7 illustrates an exemplary aortic arch implant 705 partially loaded within exemplary aortic arch delivery system 700. In some embodiments, exemplary aortic arch implant 705 comprises expandable arch support structure 710. In some embodiments, exemplary aortic arch implant 705 comprises one or more expandable branches 715. In some embodiments, one or more expandable branches 715 comprises a telescoping structure. In some embodiments, one or more expandable branches 715 are in a first configuration. For example, in some embodiments, a first expandable branch 720 is inverted, wherein at least a portion of a distal portion or a middle portion of the first expandable branch 720 is partially collapsed into a lumen of the expandable arch support structure 710. In some embodiments, one or more expandable branches 715 are in a second configuration. For example, in some embodiments, a second expandable branch 730 is crimped and folded parallel to the expandable arch support structure 710. In some embodiments, one or more expandable branches 715 are in a third configuration. For example, in some embodiments, a third expandable branch 740 is crimped and folded parallel to expandable arch support structure 710 and adjacent second expandable branch 730.

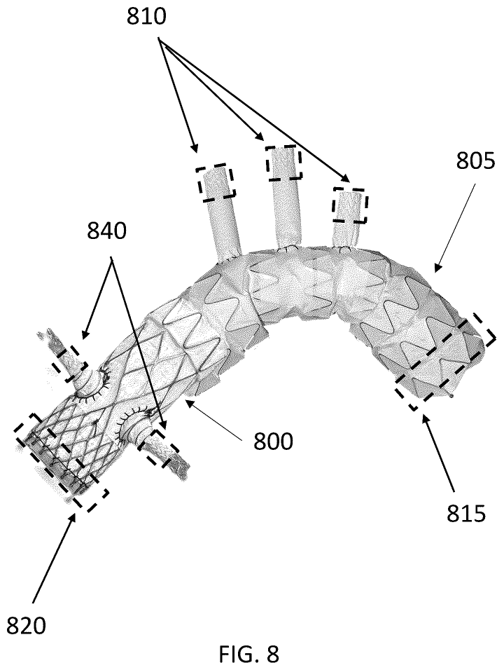

FIG. 8 illustrates an aortic root implant 800 coupled to an aortic arch implant 805. Without wishing to be bound by any particular theory, it is further believed that interlocking an expandable root support structure 830 of aortic root implant 800 to an expandable arch support structure 840 of aortic arch implant 805 advantageously permits multiple anchoring/sealing points of the prosthetic implant to the native aorta without exerting excessive force on the structures within the native aorta. For example, it is believed that anchoring/sealing point 820 anchors/seals aortic root implant 800 against an aortic root of a patient; anchoring/sealing point 840 anchors/seals one or more expandable branches of aortic root implant 800 within the right and/or left coronary ostium; anchoring/sealing point 810 anchors/seals one or more expandable branches of the aortic arch implant 805 within the brachiocephalic artery, left common carotid artery, and/or the left subclavian artery; and anchoring/sealing point 815 anchors/seals the aortic arch implant 805 against a descending aorta.

In some embodiments, the expandable support structure (e.g., expandable root/arch support structure) is capable of being expanded from a crimped state to an expanded state (e.g., upon deployment of the prosthetic implant). In some embodiments, the expandable support structure (e.g., expandable arch support structure) is configured to be placed within an aortic arch of a native aorta and is herein generally referred to as an expandable arch support structure. In some embodiments, the expandable support structure (e.g., expandable root support structure) is configured to be placed within an aortic root of a native aorta and is herein generally referred to as an expandable root support structure. In some embodiments, the expandable support structure (e.g., expandable root/arch support structure) comprises one or more expandable branches (e.g., configured to sit within a coronary ostium of the aortic root or one or more head vessels of the aortic arch, respectively). In some embodiments, the one or more expandable branches comprises a proximal portion, a middle portion, and a distal portion. In some embodiments, a proximal portion comprises an expandable support structure. In some embodiments, the expandable support structure of the proximal portion comprises a telescoping frame and/or an expandable frame. In some embodiments, the middle portion comprises an expandable support structure. In some embodiments, the expandable support structure of the middle portion comprises a telescoping frame and/or an expandable frame. In some embodiments, the distal portion comprises an expandable support structure. In some embodiments, the expandable support structure of the distal portion comprises a telescoping frame and/or an expandable frame.

In some embodiments, a prosthetic implant is configured to be placed within an aortic root of a native aorta (e.g., an aortic root implant). In some embodiments, the prosthetic implant comprises an expandable root support structure comprising one or more openings and one or more expandable branches configured to be positioned within the one or more openings. In some embodiments, the expandable root support structure is configured to be positioned within at least a portion of an ascending portion and/or an aortic root of a native aorta. In some embodiments, the expandable root support structure comprises a valved conduit positioned at a proximal end of the expandable root support structure, and a non-porous layer that is configured to contact an outer wall of the native aorta. In some embodiments, the one or more expandable branches comprises a telescoping structure comprising a proximal portion and a distal portion, wherein an inner diameter of the proximal portion is larger than an inner diameter of the distal portion. As used herein, the term “telescoping structure” refers to any structure that permits the reversible extension and collapse (e.g., retraction or inversion) of the expandable branch. In some embodiments, the one or more expandable branches is configured to be positioned within at least a portion of at least one coronary artery of an aortic root and to permit blood flow from within the expandable root support structure, through the expandable branch, and into at least one coronary artery of the aortic root.

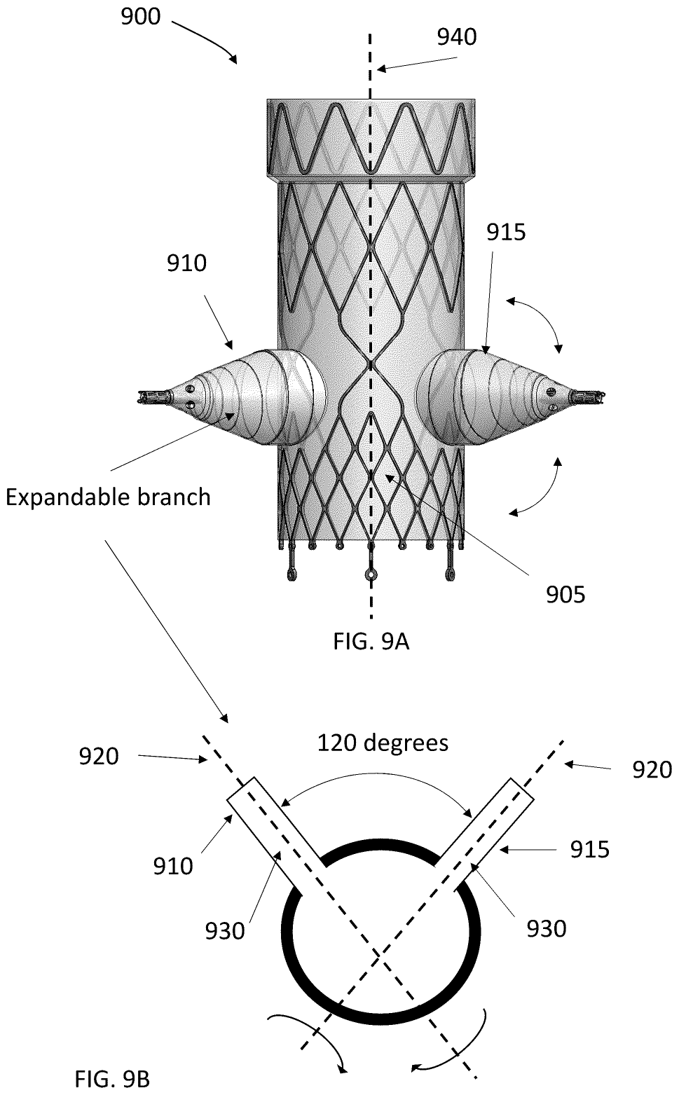

In some embodiments, a prosthetic implant is configured to be placed in a place other than the aortic root (e.g., an aortic arch implant). In some embodiments, the prosthetic implant comprises an expandable support structure comprising one or more openings and one or more expandable branches configured to be positioned within the one or more openings. In some embodiments, the one or more expandable branches comprise a telescoping structure comprising a proximal portion, a middle portion, and a distal portion. In some embodiments, the proximal portion is configured to pivot the one or more expandable branches between 0 degrees and 360 degrees, relative to a first axis (FIG. 9).

In some embodiments, an expandable branch of an expandable root support structure comprises a telescoping structure comprising a proximal portion, a middle portion, and a distal portion. Exemplary embodiments of telescoping structures are shown in FIGS. 2B-2C. For example, in some embodiments, the telescoping structure comprises an ePTFE structure (e.g., nonporous layer 250 in proximal portion 205 in FIG. 2B). In some embodiments, the telescoping structure comprises a metallic wire frame (e.g., a telescoping frame 225 in FIG. 2B) comprising a concentric cone geometry and an ePTFE layer (e.g., nonporous layer 250 in FIG. 2B) (e.g., expandable branch of expandable root support structure for placement into a coronary artery, FIGS. 1-3). Both structures permit collapse of the distal portion (e.g., distal portion 215 in FIG. 2B) and the middle portion (e.g., middle portion 210 in FIG. 2B) into the proximal portion (e.g., proximal portion 205 in FIG. 2B) of the expandable branch (e.g., expandable branch 200 in FIG. 2B). In some cases, the distal portion and/or middle portion are at least partially collapsed into a lumen of the expandable root support structure (FIG. 6).

In some embodiments, one or more expandable branches of an expandable root support structure is configured to be positioned within at least a portion of one or more vessels of an aortic root (e.g., the coronary arteries). It is believed that such configurations better anchor the implant to a native aorta and permit blood flow from within the expandable arch support structure into the coronary arteries of the native aorta (e.g., the left and/or right coronary arteries).

In some embodiments, an expandable root support structure comprises a first non-porous layer (e.g., ePTFE) (e.g., nonporous layer 250 in FIG. 2B) and is configured to contact an outer wall of the native aorta. In some embodiments, the expandable root support structure comprises one or more expandable branches comprising a telescoping structure (e.g., telescoping frame). In some embodiments, the telescoping structure comprises a tapered geometry. In some embodiments, the one or more expandable branches is configured to be positioned within at least a portion of at least one coronary artery of an aortic root of the native aorta. It is believed that, upon implantation in a subject, such configuration permit blood flow from within the expandable root support structure into at least one coronary artery of the aortic root of the native aorta.

Additionally, in some embodiments, a proximal end of an expandable root support structure comprises a valved conduit (e.g., valved conduit 315 comprising tricuspid valve 335 in FIG. 3B). In some embodiments, the valved conduit comprises a bovine aortic valve. In some embodiments, the valved conduit comprises a porcine tissue valve. In some embodiments, the valved conduit at the proximal end of the expandable root support structure is removable. In some embodiments, the proximal end of the expandable root support structure comprises a prosthetic aortic valve frame. In some embodiments, the prosthetic aortic valve frame comprises a bridge valve. In some embodiments, the prosthetic aortic valve frame comprises a destination valve. In some embodiments, the prosthetic aortic valve frame is configured to receive the aortic valve implant. In some embodiments, the prosthetic aortic valve frame does not comprise a valve. In some embodiments, the aortic valve frame reversibly receives the aortic valve implant. In some embodiments, the aortic valve frame irreversibly receives the aortic valve implant. In some embodiments, a proximal end of the prosthetic aortic valve frame is flared and extends into a left ventricular outflow track (LVOT), thereby at least partially anchoring the prosthetic implant to the native aorta.

In some embodiments, a proximal end of an expandable root support structure is in contact with but not directly adhered and/or grafted to the native aorta upon deployment in the native aorta.

In some embodiments, a prosthetic implant is configured to be positioned within an aortic root and an aortic arch of a subject in need thereof. In some embodiments, a prosthetic implant comprises an expandable root support structure (e.g., configured to be positioned within an aortic root of a native aorta) interlocked with an expandable arch support structure (e.g., configured to be positioned within at least a portion of the ascending aorta and continuing through an aortic arch and into at least a portion of a descending aorta of a native aorta). In some embodiments, the expandable root support structure comprising one or more openings, and one or more expandable branches configured to be positioned within the one or more openings is sized and configured to be positioned within the ascending portion of the native aorta. In some embodiments, the expandable root support structure comprises a valved conduit positioned at a proximal end of the expandable end of the expandable root support structure and a non-porous layer that is configured to contact an outer wall of at least a portion of a native aorta. In some embodiments, the expandable root support structure comprises one or more expandable branches comprising a telescoping structure (e.g., comprises a telescoping frame), and optionally, a tapered geometry. In some embodiments, the one or more expandable branches comprises a proximal portion, a middle portion, and a distal portion. In some embodiments, the one or more expandable branches is configured to be positioned within at least a portion of at least one coronary artery of an aortic root and to permit blood flow from within the expandable root support structure into at least one coronary artery of the aortic root.

As described above, in some embodiments, an expandable root support structure is interlocked with an expandable arch support structure. In some embodiments, any suitable method of interlocking the two support structures known in the art is used by the skilled artisan. In some embodiments, the distal end of the expandable root structure is configured to engage a proximal end of the expandable arch support structure.

In some embodiments, an expandable arch support structure is configured to be positioned within at least a portion of the ascending aorta, an aortic arch, and at least a portion of a descending aorta of a subject in need thereof. In some embodiments, the expandable arch support structure comprises one or more openings and one or more expandable branches configured to be positioned within the one or more openings (FIG. 4A-4C). In some embodiments, the expandable arch support structure is configured to be positioned within at least a portion of a descending portion of a native aorta. In some embodiments, the expandable arch support structure comprises a nonporous layer configured to contact an outer wall of the native aorta. In some embodiments, the nonporous layer comprises ePTFE. In some embodiments, the expandable arch support structure comprises one or more changes in a cross-sectional dimension within the body of the expandable arch support structure. For example, in some cases, a distal portion and a proximal portion of the expandable arch support structure have a first cross-sectional dimension different (e.g., larger) than a middle portion of the expandable arch support structure.

In some embodiments, an expandable branch of an expandable arch support structure comprises a telescoping structure comprising a proximal portion, a middle portion, and a distal portion. Exemplary embodiments of telescoping structures are shown in FIG. 4C. For example, in some embodiments, the telescoping structure comprises an ePTFE structure (e.g., proximal portion shown in FIG. 4C). In some embodiments, the telescoping structure comprises a metallic wire frame (e.g., distal expandable frame 475 in distal portion 470 of expandable branch 420 shown in FIG. 4C) comprising a tubular geometry (e.g., expandable branch of expandable arch support structure for placement into at least one head vessel of the aortic arch). Both structures permit collapse of the distal portion and the middle portion into the proximal portion of the expandable branch. In some cases, the distal portion and/or middle portion are at least partially collapsed into a lumen of the expandable arch support structure, as shown, for example, in FIG. 7 (e.g., shows expandable branch for placement into the brachiocephalic artery is inverted).

In some embodiments, one or more expandable branches of an expandable arch support structure is configured to be positioned within at least a portion of one or more vessels of an aortic arch of the native aorta. It is believed that such configurations better anchor the implant to a native aorta and permit blood flow from within the expandable arch support structure into the vessels of the aortic arch of the native aorta (e.g., brachiocephalic artery, left common carotid artery, and left subclavian artery).

Without wishing to be bound by any particular theory, it is further believed that interlocking an expandable root support structure to an expandable arch support structure advantageously permits multiple anchoring/sealing points of the prosthetic implant to the native aorta without exerting excessive force on the structures within the native aorta (FIG. 8).

In some embodiments, a prosthetic implant, as contemplated herein, comprises one or more expandable branches of an expandable root support structure. In some embodiments, the one or more expandable branches are configured to be placed within a coronary artery (e.g., right and/or left coronary artery). Those of skill in the art will understand and appreciate that the anatomical features of the coronaries may be, in some cases, different, or the same, as the vessels in the aortic arch. Thus, in some embodiments, the one or more expandable branches of the expandable root support structure have a different geometry and/or configuration than the one or more branches of an expandable arch support structure. For example, in some embodiments, one or more expandable branches of the expandable root support structure comprise a proximal portion, a middle portion, and a distal portion (as shown in FIG. 2). In some embodiments, the proximal portion of the one or more expandable branches of the expandable root support structure does not comprise a metallic wire frame (e.g., does not comprise a telescoping wire frame but may comprise a telescoping polymer frame). In some embodiments, the middle portion of the one or more expandable branches of the expandable root support structure comprises a metallic wire frame (e.g., a telescoping metallic wire frame). The metallic wire frame, in some embodiments, comprises a nonporous layer (e.g., ePTFE covering). In some embodiments, the distal portion of the one or more expandable branches of the expandable root support structure comprises an expandable support structure (e.g., a distal expandable frame). In some embodiments, expandable support structure (e.g., a distal expandable frame) of the distal portion comprises an ePTFE covering (e.g., nonporous layer), optionally, wherein the ePTFE covering, comprises one or more pores. In some embodiments, when the expandable support structure (e.g., distal expandable frame) of the distal portion is in a crimped geometry, the one or more pores in the ePTFE covering (e.g., nonporous layer) are open (e.g., patent). It is believed that such configurations may be, in some cases, useful, for example, to preserve coronary blood flow during anchoring of the expandable root support structure to the coronary arteries. In some embodiments, when the expandable support structure (e.g., distal expandable frame) of the distal portion is an expanded geometry, the one or more pores in the ePTFE covering (e.g., nonporous layer) are closed. It is believed that such configurations may be, in some cases, useful, for example, to prevent leakage of blood from the expandable branches within the coronary arteries.

In some embodiments, one or more expandable branches of an expandable root support structure are also configured to collapse within at least a portion of the implant. Again, it is believed that such configurations are advantageous, for example, for delivering the implant to the desired anatomical location (FIG. 6). Thus, in some embodiments, when a metallic wire frame (e.g., telescoping frame) of a middle portion of the one or more expandable branches is in a collapsed state, the middle portion is configured to sit within a proximal portion of the one or more expandable branches. In some embodiments, when an expandable support structure (e.g., distal expandable frame) of a distal portion of the one or more branches is in a crimped state and the middle portion is in a collapsed state, the distal and middle portions are configured to fit within the proximal portion of the expandable branch and/or within a lumen of the expandable root support structure.

As described above, in some embodiments a prosthetic implant comprises an expandable branch of an expandable arch support structure. In some embodiments, the proximal portion of the one or more expandable branches does not comprise a telescoping metallic wire frame or an expandable support structure (e.g., an expandable metallic wire frame). In some embodiments, the proximal portion of the one or more expandable branches comprises a telescoping polymer frame, such as for example, a nonporous layer configured into a tubular or cylindrical shape. In some embodiments, the proximal portion of the one or more expandable branches comprises a nonporous layer (e.g., ePTFE) configured into a tubular or cylindrical shape. In some embodiments, the nonporous layer is ePTFE. In some embodiments, the middle portion of the one or more expandable branches comprises a telescoping frame and/or an expandable support structure (e.g., a distal expandable frame such as an expandable metallic wire frame). In some embodiments, the expandable branch is at least partially covered by a nonporous layer (e.g., ePTFE). In some embodiments, the expandable branch is at least partially covered by an ePTFE layer. In some embodiments, the distal portion of the one or more expandable branches comprises an expandable support structure (e.g., a distal expandable frame such as an expandable metallic wire frame). In some embodiments, the distal portion of the one or more expandable branches comprising the expandable support structure is not covered in a non-porous layer (e.g., ePTFE). In some embodiments, the distal portion of the one or more expandable branches comprising the expandable support structure (e.g., a distal expandable frame) is not covered in ePTFE.

As with the expandable root support structure, one or more expandable branches of an expandable arch support structure is also configured to collapse within at least a portion of the implant. It is believed that such configurations are advantageous, for example, for delivering the implant to the desired anatomical location. For example, in some embodiments, when a middle and distal portions comprises an expandable support structure (e.g., telescoping and/or expanding frames) and the proximal portion comprises an ePTFE telescoping structure (e.g., nonporous layer configured into a tubular structure) and when the middle portion and the distal portion of the one or more expandable branches are in a crimped state, the middle portion and distal portions are configured to collapse into the proximal portion of the one or more expandable branches. In some embodiments, the proximal portion, middle portion, and the distal portions may also collapse, at least partially, into a lumen of the expandable arch support structure.

In some embodiments, one or more expandable branches of an expandable root support structure and/or an expandable arch support structure are configured to have similar, or different, structures and/or configurations. For example, in some embodiments, a proximal portion of the one or more expandable branches is configured to be coupled to the middle portion of the one or more expandable branches; and the middle portion of the one or more expandable branches is configured to be coupled to the distal portion of the one or more expandable branches.

FIG. 9 shows another embodiment of an exemplary aortic root implant 900 comprising expandable root support structure 905, a first expandable branch 910, and a second expandable branch 915. In some embodiments, first expandable branch 910 and second expandable branch 915 are configured to pivot radially between 0 degrees and 360 degrees, relative to a first axis 920. In some embodiments, the first expandable branch 910 and second expandable branch 915 are configured to pivot laterally between 90 degrees and −90 degrees, relative to a second axis 940. In some embodiments, the first axis 920 runs parallel to an elongate central passageway 930 defined by the one or more expandable branches (e.g., a lumen). In some embodiments, the second axis 940 runs perpendicular to the elongate central passageway defined by the one or more expandable branches (e.g., the second axis is parallel to the expandable root/arch support structure). In some embodiments, first expandable branch 910 and second expandable branch 915 are separated by about 120 degrees, and/or generally corresponds to the anatomical positioning of the right and left coronary ostia in the aortic root.

In some embodiments, one or more expandable branches of an expandable root support structure and/or an expandable arch support structure are configured to pivot radially between 0 degrees and 360 degrees, relative to a first axis. In some embodiments, the first axis runs parallel to an elongate central passageway defined by the one or more expandable branches (e.g., a lumen). In some embodiments, one or more expandable branches of an expandable root support structure and/or an expandable arch support structure are configured to pivot laterally between 90 degrees and −90 degrees, relative to a second axis. In some embodiments, the second axis runs perpendicular to the elongate central passageway defined by the one or more expandable branches.

In some embodiments, one or more expandable branches of an expandable root support structure and/or an expandable arch support structure are configured to be in an extended state. In some embodiments, the one or more expandable branches of an expandable root support structure and/or an expandable arch support structure are configured to be in a collapsed state.

In some embodiments, one or more expandable branches of an expandable root support structure and/or an expandable arch support structure are configured to have a conical geometry. In some embodiments, in an expanded state, an inner diameter of the proximal portion of the one or more expandable branches is larger than an inner diameter of the distal portion of the one or more expandable branches.

In some embodiments, one or more expandable branches of an expandable root support structure and/or an expandable arch support structure is configured to have a cylindrical geometry. In some embodiments, in an expanded state, an inner diameter of the proximal portion of the one or more expandable branches is approximately the same as an inner diameter of the distal portion of the one or more expandable branches.

In some embodiments, one or more expandable branches of an expandable root support structure and/or an expandable arch support structure comprise a metallic wire frame (e.g., a proximal portion, a middle portion, and/or a distal portion) and/or expandable support structure (e.g., a proximal portion, a middle portion, and/or a distal portion) covered by a nonporous layer (e.g., ePTFE). In some embodiments, the nonporous layer comprises ePTFE.

In some embodiments, one or more expandable branches of an expandable root support structure and/or an expandable arch support structure comprise a metallic wire frame (e.g., a proximal portion, a middle portion, and/or a distal portion) and/or expandable support structure (e.g., a proximal portion, a middle portion, and/or a distal portion) covered by a porous layer. In some embodiments, the porous layer comprises ePTFE.

As described above, a primary function of the one or more expandable branches of an expandable root support structure is to anchor to the coronary arteries and to permit blood flow from within the expandable root support structure into a left coronary artery and/or right coronary artery. Thus, in some embodiments, the expandable root support structure comprises a first expandable branch configured to be positioned within at least a portion of a left coronary artery. In some embodiments, the expandable root support structure comprises a first expandable branch configured to be positioned within at least a portion of a right coronary artery. In some embodiments, the expandable root support structure comprises a first expandable branch configured to be positioned within at least a portion of the left coronary artery and the second expandable branch is configured to be positioned within at least a portion of the right coronary artery. In some embodiments, the distal portion of the first expandable branch and the distal portion of the second expandable branch each comprise an expandable support structure, that when expanded, is configured to apply a radially outward force to a coronary artery, thereby anchoring the implant to the native aorta. In some embodiments, the first expandable branch and the second expandable branch of the expandable root support structure is configured to keep open a right and/or left coronary ostium. In some embodiments, the expandable root support structure comprises a first expandable branch positioned e.g., 120 degrees (e.g., from 90 degrees to 170 degrees) from a second expandable branch, relative to a second axis. In some embodiments, the first expandable branch and/or second expandable branch are positioned at least 15 mm (e.g., at least 5-25 mm) above the proximal end of the expandable root support structure.

In an alternative set of embodiments, an expandable root support structure is anchored to the aortic root using an expandable anchoring structure (e.g., as opposed to anchoring to the coronary vessels). In some embodiments, the expandable anchoring structure comprises an expandable trilobe structure. In some embodiments, at least a portion of the expandable trilobe structure applies a radially outward force to an aortic sinus thereby anchoring the prosthetic implant to the native aorta. In some embodiments, the expandable trilobe structure further comprises a trilobe structure comprising three lobes, wherein each of the three lobes is sized and configured to conform to a curvature of the aortic sinus when the expanded anchoring structure is expanded. In some embodiments, each of the three lobes comprises one or more apex at a distal end of the expandable trilobe structure configured to be positioned adjacent to an aortic valve annulus of the patient. In some embodiments, the trilobe structure is configured to expand to engage an inner wall of the aortic sinus separately from expansion of the expandable root support structure, and the prosthetic implant is configured to sequentially deploy the trilobe structure before the expandable root support structure. In some embodiments, the one or more native leaflets are secured between the proximal end of the expandable root support structure and the one or more backstop elements of the expandable anchoring structure thereby at least partially anchoring the prosthetic implant to the native aorta. In some embodiments, the one or more backstop elements of the expandable anchoring structure are sized and configured to prevent one or more of the native leaflets from blocking flow into one or more coronary arteries by ensuring the one or more native leaflets cannot expand beyond the backstops and block one or more ostia of a right coronary artery and/or left coronary artery. In some embodiments, the expandable anchoring structure engages one or more aortic sinuses thereby anchoring the prosthetic implant and/or promoting a seal at within the region just above a sinotubular junction in the native aorta. In some embodiments, the one or more expandable anchoring structures, configured to extend within a left and a right aortic sinus, is at least partially uncovered such that the left and right coronary ostia remain uncovered by the prosthetic implant when in use. In some embodiments, the expandable anchoring structure further comprises a metallic wire frame.

Likewise, a primary function of the one or more expandable branches of an expandable arch support structure is to anchor to the vessels of the aortic arch and to permit blood flow from within the expandable arch support structure into at least the left subclavian artery, the left common carotid artery, and/or the brachiocephalic artery of the native aorta. Thus, in some embodiments, the expandable arch support structure comprises a first expandable branch configured to be positioned within at least a portion of the left subclavian artery. In some embodiments, the expandable arch support structure comprises a first expandable branch configured to be positioned within at least a portion of the left common carotid artery. In some embodiments, the expandable arch support structure comprises a first expandable branch configured to be positioned within at least a portion of the brachiocephalic artery. In some embodiments, the expandable arch support structure comprises a first expandable branch configured to be positioned within at least a portion of the left common carotid artery and a second expandable branch configured to be positioned within at least a portion of the brachiocephalic artery. In some embodiments, the expandable arch support structure comprises a first expandable branch configured to be positioned within at least a portion of the left common carotid artery and a second expandable branch configured to be positioned within at least a portion of the subclavian artery. In some embodiments, the expandable arch support structure comprises a first expandable branch configured to be positioned within at least a portion of the brachiocephalic artery and the second expandable branch configured to be positioned within at least a portion of the subclavian artery. In some embodiments, the expandable arch support structure comprises a first expandable branch configured to be positioned within at least a portion of the brachiocephalic artery, a second expandable branch is configured to be positioned within at least a portion of the left common carotid artery, and a third expandable branch is configured to be positioned within at least a portion of the subclavian artery.

In some embodiments, an expandable root support structure and/or an expandable arch support structure comprises a non-porous layer, for example, to wall off a dissection. In some embodiments, the non-porous layer is configured to expand and, upon expansion, apply a radially outward force to the ascending aorta thereby forming a seal against the internal surface of the aorta and/or anchoring the prosthetic implant to the native aorta. In some embodiments, the non-porous layer expands due to a blood hydrostatic pressure created by blood flowing through an intraluminal space formed between the non-porous layer and the porous layer. In some embodiments, the non-porous layer contacts the inner wall of the native aorta between a brachiocephalic trunk and the sinotubular junction of the native aorta. In some embodiments, the non-porous layer is configured to be positioned across at least a portion of a dissection. In some embodiments, the non-porous layer is configured to seal around at least a portion of the dissection. In some embodiments, the non-porous layer is configured to prevent blood flow through the dissection. In some embodiments, the non-porous layer comprises an opening to allow blood to flow from within the expandable support structure, through the opening, and into the carotid and the subclavian arteries of the native aorta. In some embodiments, the non-porous layer comprises a second coating. In some embodiments, the second coating prevents adhesion of at least one or more component of blood. In some embodiments, the second coating comprises a therapeutic agent.

In some embodiments, an expandable root support structure and/or an expandable arch support structure comprises a porous layer, for example, in place of the one or more expandable branches (e.g., an expandable arch support graft comprising zero, one, or two expandable branches). As one of skill in the art will appreciate, the porous layer is intended to permit blood flow from inside the expandable arch support graft and into the vessels of the aortic arch. Any suitable porous layer known to the skilled artisan may be, in some cases, used in the expandable arch support structures described herein. In some embodiments, the porous layer has an areal density of between 0-300 g/m\hat{ }2. In some embodiments, the porous layer has an average areal density of between 50-300 g/m\hat{ }2. In some embodiments, the second porous layer has an average 0.01-5.00 mm. In some embodiments, the porous layer has an average pore size of between 0.01-2.00 mm. In some embodiments, the second porous layer has a thickness of between 0-200 microns. In some embodiments, the porous layer has a thickness of between 10-200 microns. In some embodiments, the porous layer comprises a coating. In some embodiments, the porous layer prevents adhesion of at least one or more components of blood to the porous layer. In some embodiments, the coating further comprises a therapeutic.

The prosthetic implants of the present disclosure, may according to some embodiments, comprise any combination of structures and/or configurations disclosed herein. For example, in some embodiments, the expandable root support structure comprises a metallic frame that extends continuously from the proximal end to a distal end of the expandable root support structure and that is continuous with a metallic frame of an expandable anchoring structure. In some embodiments, the expandable root support structure and the expandable anchoring structure comprise separate frames. In some embodiments, the expandable root support structure and the expandable anchoring structure are formed from a single continuous wire. In some embodiments, the expandable root support structure comprises a metal frame that is continuous with a metallic frame of an expandable arch support structure. In some embodiments, however, the expandable root support structure and the expandable arch support structure comprise separate frames.

In some embodiments, the expandable arch support structure is sized and configured to be positioned within at least a portion of the descending portion of the native aorta. In some embodiments, the expandable arch support structure is configured to permit blood flow from within the expandable arch support structure, through a porous layer, and into one or more carotid arteries and/or subclavian arteries of the native aorta. In some embodiments, the expandable arch support structure, comprising the porous layer, is configured to substantially cover the descending aorta to a brachiocephalic trunk of the native aorta and an expandable root support structure, comprising a non-porous layer, is configured to engage a wall of the ascending aorta on opposite sides of a tear of the dissection.

In some embodiments, an expandable arch support structure comprises a metallic frame that extends continuously from a proximal end to a distal end of the expandable arch support structure and that is continuous with the metallic frame of an expandable anchoring structure. In some embodiments, the expandable arch support structure and the expandable anchoring structure comprise separate frames.

In some embodiments, an expandable root support structure, an expandable anchoring structure, and an expandable arch support structure comprise separate frames. In some embodiments, the expandable root support structure, the expandable anchoring structure, and the expandable arch support structure are formed from a single continuous wire.

In some embodiments, an expandable arch support structure and an expandable anchoring structure are formed from a single continuous wire.

In some embodiments, when loaded into a delivery device the one or more expandable branches configured to be positioned within the left subclavian is in a crimped position and sits parallel to a distal portion of the expandable arch support structure.

In some embodiments, an expandable arch support structure is pre-formed with a curvature to conform to an aortic arch of the native aorta.

In some embodiments, an expandable root support structure is configured to be coupled to an expandable arch support structure, thereby anchoring the expandable root support structure to the expandable arch support structure. In some embodiments, the expandable root support structure configured to be positioned within an ascending portion of the native aorta is coupled to an expandable arch support structure configured to be positioned within at least a portion of the descending portion of the native aorta. In some embodiments, an expandable arch support structure does not apply a radially outward force against the native aorta.

The skilled artisan will understand that a prosthetic implant as disclosed herein may be, in some cases, used to treat any suitable disease or condition known to the skilled artisan. For example, in some embodiments, the implant is configured to treat an aortic dissection. In some cases, the aortic dissection is a Type A aortic dissection. Treatment of other conditions is also contemplated herein, according to other embodiments. For example, in some embodiments, the implant is configured to treat an intramural hematoma. Additionally, or alternatively, the implants disclosed herein may be, in some cases, used to treat a thoracic aortic aneurysm.

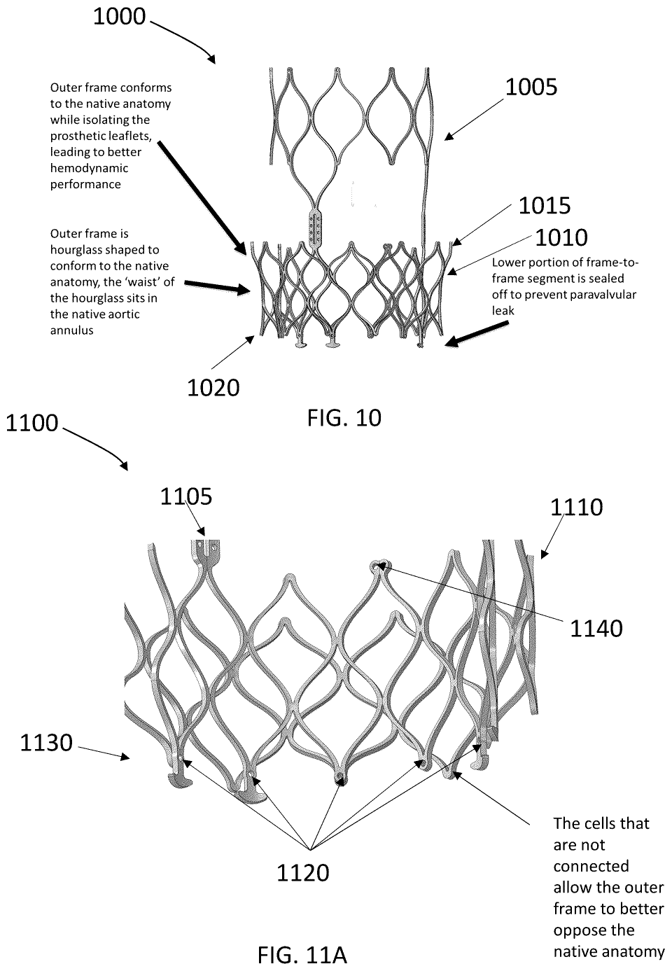

In some embodiments, an aortic root implant comprises a dual frame, the dual frame comprising an inner frame and an outer frame. FIG. 10 shows an exemplary dual framed aortic root implant 1000, as contemplated herein. In some embodiments, inner frame 1005 extends out from a distal portion 1015 of the main body of outer frame 1010. In some embodiments, inner frame 1005 extends out from a proximal portion 1020 of the main body of outer frame 1010.

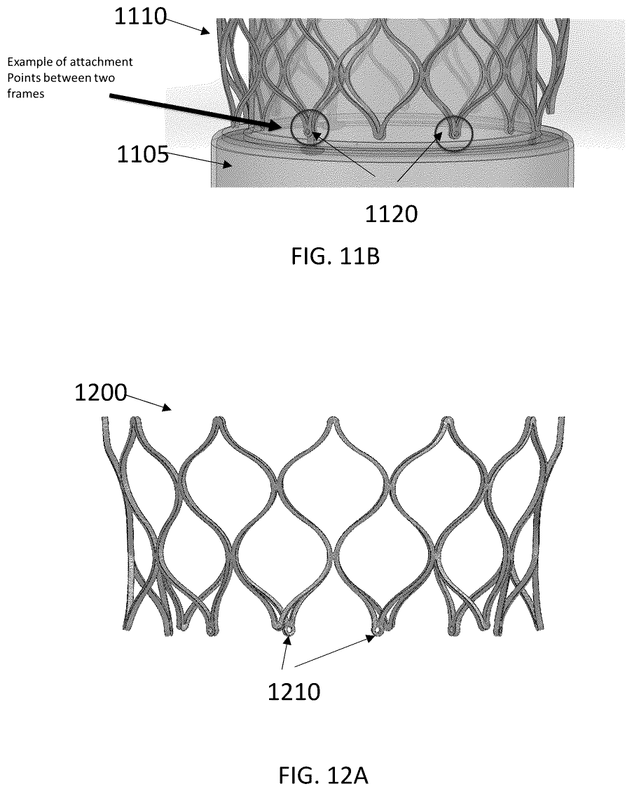

In some embodiments, a dual frame aortic root implant comprises an outer frame and/or an inner frame comprising a plurality of anchors points. For example, FIG. 11A shows an exemplary dual frame aortic root implant 1100, comprising inner frame 1105 and outer frame 1110. In some embodiments, inner frame 1105 comprises a plurality of first anchor points 1120 at a proximal portion 1130 of the main body of outer frame 1110 configured to anchor the inner frame 1105 to outer frame 1110. In some embodiments, inner frame 1105 comprises a plurality of second anchor points 1140 at an upper part of inner frame 1105 configured to anchor inner frame 1105 to one or more expandable branches. FIG. 11B shows a computer aided drawing (CAD) of inner frame 1105 anchored to outer frame 1110 via a plurality of first anchor points 1120.

In some embodiments, the outer frame is operably linked to the inner frame. In some embodiments, the plurality of first anchor points 1120 is used to anchor the outer frame 1110 to the inner frame 1105. For example, in some embodiments, a suture may be passed through the plurality of anchor points 1120 and used to anchor inner frame 1105 to outer frame 1110 via a suture knot. In some embodiments, any suitable suture material and/or suture knot known to the skilled artisan may be used to secure the inner frame to the outer frame. In some embodiments, the frames are sutured together at every other cell, as shown in FIG. 11A.

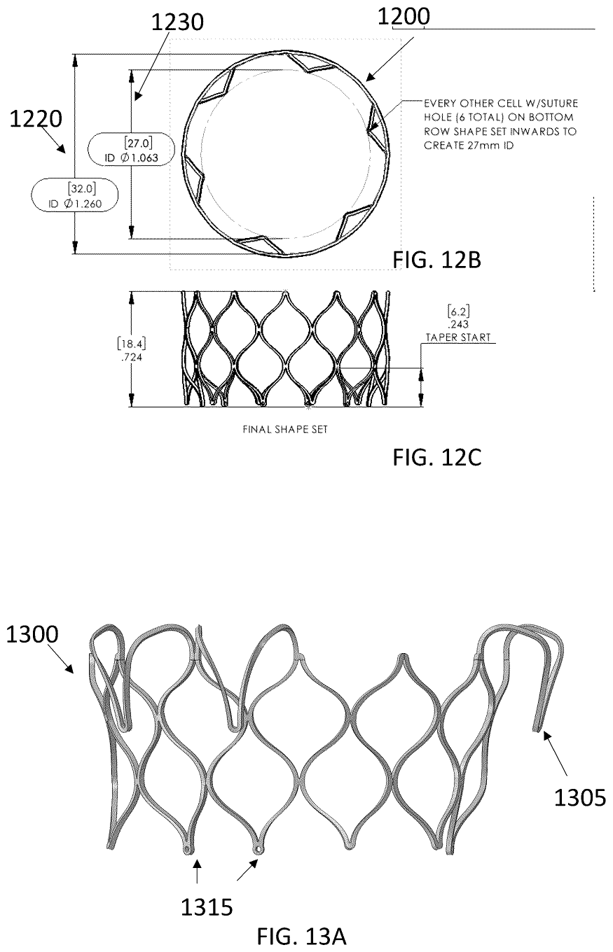

FIG. 12A-12C shows exemplary outer frame 1200 comprising a plurality of first anchor points 1210. In some embodiments, exemplary outer frame 1200 has first inner diameter 1220. In some embodiments, every other anchor point is set inwards to create a second inner diameter 1230.

In some embodiments, the outer frame has a total height of between 15 mm and 25 mm. In some embodiments, the outer frame has a total height of greater than or equal to 15 mm, greater than or equal to 16 mm, greater than or equal to 17 mm, greater than or equal to 18 mm, greater than or equal to 19 mm, greater than or equal to 20 mm, greater than or equal to 21 mm, greater than or equal to 22 mm, greater than or equal to 23 mm, greater than or equal to 24 mm, or greater than or equal to 25 mm. In some embodiments, the outer frame has a total height of less than or equal to 25 mm, less than or equal to 24 mm, less than or equal to 23 mm, less than or equal to 22 mm, less than or equal to 21 mm, less than or equal to 20 mm, less than or equal to 19 mm, less than or equal to 18 mm, less than or equal to 17 mm, less than or equal to 16 mm, or less than or equal to 15 mm. Combinations of the above recited ranges are also possible in some embodiments. For example, in some embodiments, the outer frame has a total height of greater than or equal to 15 mm and less than or equal to 25 mm. In some embodiments, the outer frame has a total height of about 18.4 mm.

In some embodiments, the outer frame has an inner diameter of between 25 mm and 35 mm. In some embodiments, the outer frame has an inner diameter of greater than or equal to 25 mm, greater than or equal to 26 mm, greater than or equal to 27 mm, greater than or equal to 28 mm, greater than or equal to 29 mm, greater than or equal to 30 mm, greater than or equal to 31 mm, greater than or equal to 32 mm, greater than or equal to 33 mm, greater than or equal to 34 mm, or greater than or equal to 35 mm. In some embodiments, the inner diameter is less than or equal to 35 mm, less than or equal to 34 mm, less than or equal to 33 mm, less than or equal to 32 mm, less than or equal to 31 mm, less than or equal to 30 mm, less than or equal to 29 mm, less than or equal to 28 mm, less than or equal to 27 mm, less than or equal to 26 mm, or less than or equal to 25 mm. Combinations of the above recited ranges are also possible in some embodiments. For example, in some embodiments, the outer frame has an inner diameter of greater than or equal to 25 mm and less than or equal to 32 mm. In some embodiments, the outer frame has a first inner diameter and a second inner diameter, as shown in FIG. 12B. In some embodiments, the first inner diameter is about 32 mm. In some embodiments, the second inner diameter is about 27 mm.

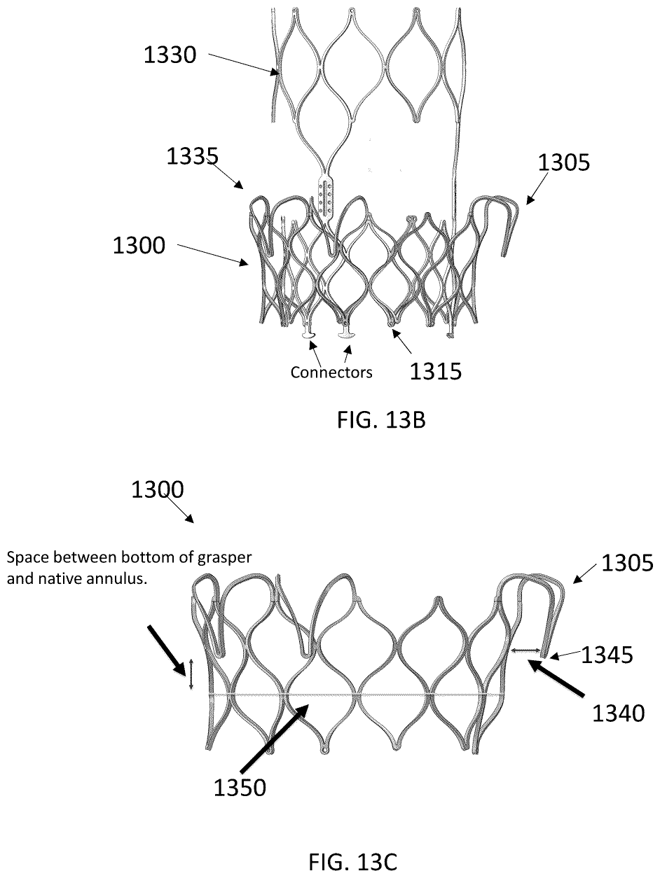

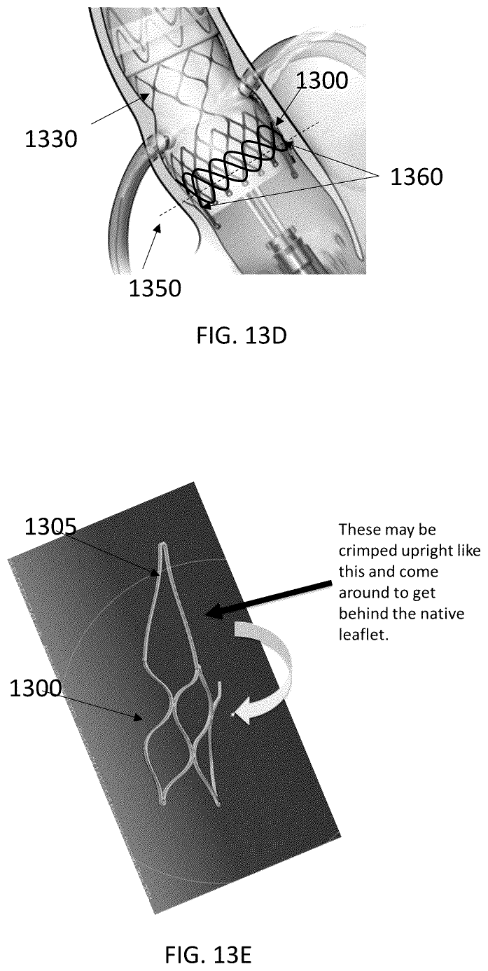

FIGS. 13A-13E illustrate various embodiments of a dual framed aortic root implant. For example, FIG. 13A shows exemplary outer frame 1300 comprising a plurality of leaflet anchors 1305 at a first end 1310 of exemplary outer frame 1300 and a plurality of anchor points 1315 at a second end 1320 of exemplary outer frame 1300, the second end 1320 at an opposite end of exemplary outer frame 1300 relative to first end 1310. In some embodiments, exemplary outer frame 1300 comprises plurality of leaflet anchors 1305. In some embodiments, plurality of leaflet anchors 1305 are positioned at a distal end 1335 of the exemplary outer frame 1300. In some embodiments, the plurality of leaflet anchors 1305 are configured to anchor a dual framed aortic root device within a native aortic root of a subject. In some embodiments, plurality of leaflet anchors 1305 comprises a “n-shaped” geometry, as shown illustratively in FIGS. 13A-13C. In some embodiments, the “n-shaped” geometry enables the plurality of leaflet anchors 1305 to be positioned behind the native leaflets, thus anchoring the device to the aortic root. For instance, plurality of leaflet anchors 1305 may extend from and be contiguous with the outer frame and may be folded in an orientation such that a portion of the plurality of leaflet anchors 1305 is substantially parallel to at least a portion of the body of the exemplary outer frame 1300. Other leaflet anchor geometries are also contemplated in some embodiments. For example, in some embodiments, the leaflet anchor comprises a pincer-like geometry. In some embodiments, the “pincer-like” geometry pinches the native leaflet in between a tip of the leaflet anchor and the body of the outer frame.

FIG. 13B shows exemplary outer frame 1300 anchored to inner frame 1330. As shown illustratively in FIG. 13C, in some embodiments, gap 1340 is present between the body of the outer frame 1300 and tip 1345 of plurality of leaflet anchors 1305. In some embodiments, there is no gap between the body of the outer frame and a tip of the leaflet anchor. In some embodiments, tip 1345 of the plurality of leaflet anchors 1305 is a set distance away from the base of the annulus, such that tip 1345 of the plurality of leaflet anchors 1305 is configured not to touch a base of a native annulus of a native heart valve (e.g., native annular plane 1350). In some embodiments, the distance is between 1 and 10 mm.

In some embodiments, a portion of the outer frame is configured to contact the native annulus. For example, in some embodiments, a portion of the outer frame 1300 (e.g., a central portion as shown in FIG. 13D) is configured to contact the annulus at a native annular plane 1350. In some embodiments, a seal 1360 is formed upon contact of the outer frame 1300 with the native annulus at native annular plane 1350, as shown in FIG. 13D (e.g., to prevent black flow of blood).

In some embodiments, the outer frame is at least partially covered by a nonporous layer (e.g., an ePTFE layer). The nonporous layer may be any nonporous layer described anywhere herein. In some embodiments, the nonporous layer comprises an ePTFE layer. In some embodiments, the entire outer frame is covered in the nonporous layer. However, the skilled artisan will understand that the aforementioned embodiments are not meant to be limiting, and that in some embodiments, the nonporous layer may comprise one or more openings. Other aspects of the disclosure relate to methods of anchoring the dual frame aortic implant within a native aorta. In some embodiments, the dual frame aortic implant is loaded within a delivery device configured to delivery said aortic implants, such as those disclosed in U.S. Patent Application No.: 63/575171, entitled “Arch Graft Implants and Related Methods” filed on Apr. 5, 2024 and U.S. Patent Application No.: 63/575133, entitled “Aortic Root Implants and Related Systems and Method” filed on Apr. 5, 2024, both of which are herein incorporated by reference in their entirety.

In some embodiments, the dual frame aortic implant is placed within a sheath of a delivery device. Those of skill in the art will understand and appreciate that a leaflet anchor of an outer frame maybe crimped into one or more configurations prior to loading into the sheath. This is possible, for example, when using nitinol wire frames as the outer frame material because nitinol is a shape memory alloy. Thus, in some embodiments, the outer frame may be loaded into the sheath of the delivery device with the leaflet anchor in a bent position (FIG. 13A).

Under these conditions, when the outer frame is released from the sheath (e.g., during deployment) the leaflet anchor deploys in a bent position. This permits the leaflet anchors to drop down behind the native leaflet as the implant is lowered into position. Alternatively, in some embodiments, the outer frame 1300 may be loaded into the sheath of the delivery device with the plurality of leaflet anchors 1305 crimped in an upright position (e.g., from a bent position) (FIG. 13E). The skilled artisan will understand that crimping the plurality of leaflet anchors 1305 in an upright position puts the anchors under tension. Under these conditions, when the outer frame 1300 is released from the sheath, (e.g., during deployment) the crimped upright leaflet anchor snaps into a bent geometry (as shown in FIG. 13A), thus pinching the native leaflet between the leaflet anchor and the body of the outer frame.

Accordingly, in some embodiments, a dual frame aortic implant comprises an outer frame and an inner frame. In some embodiments, the inner frame is operably linked to the outer frame. In some embodiments, the inner frame comprises an expandable root support structure comprising one or more openings and one or more expandable branches configured to be positioned within the one or more openings and operably linked to the expandable root support structure. In some embodiments, the expandable root support structure is configured to be positioned within at least a portion of an ascending portion of a native aorta. In some embodiments, the expandable root support structure comprises a valved conduit positioned at a proximal end of the expandable root support structure, and a non-porous layer that is configured to contact an outer wall of the native aorta. In some embodiments, the one or more expandable branches comprises a telescoping structure comprising a proximal portion and a distal portion, wherein an inner diameter of the proximal portion is larger than an inner diameter of the distal portion. In some embodiments, the one or more expandable branches is configured to be positioned within at least a portion of at least one coronary artery of an aortic root and to permit blood flow from within the expandable root support structure and into at least one coronary artery of the aortic root.

In some embodiments, the outer frame of a dual framed aortic implant enables sealing of the implant against a native heart annulus (e.g., calcified or non-calcified aortic annulus). In some embodiments, the outer frame of the dual framed aortic implant enables anchoring of the devices against the native heart annulus (e.g., calcified or non-calcified aortic annulus).

In some embodiments, an implant (e.g., aortic root/arch implant) as disclosed herein comprises one or more frames. In some embodiments, the frame is a part of an expandable root/arch support structure. In some embodiments, the frame is a telescoping frame. In some embodiments, the frame is a distal expandable frame. As used herein, the terms “expandable support structure,” “expandable root/arch support structure,” and/or “distal expandable frame” are synonymous and refer to any frame that permits radial expansion and contraction (e.g., crimping) about a central axis. Additionally, as used herein, the term “telescoping frame” refers to any frame that permits extension, inversion, and/or rotation of at least a portion of an expandable branch about an axis.

The one or more frames of the present invention, according to some embodiments, is a wire frame or wire coil with a zigzag or Z-shaped pattern along a cylindrical portion of the coil. The skilled artisan will understand, however, that frame may comprise other patterns suitable for treating an aortic dissection. For example, in some embodiments, the frame may have a braided configuration, a sine wave pattern, or a trilobe pattern. In some embodiments, the frame is a coiled wire forming a wire frame, such as, for example, a coiled ribbon. In some embodiments, the frame is formed from one or more of a metal (e.g., stainless steel, nitinol, or the like), a polymer (e.g., ePTFE), a biological material, a bio-absorbable material, and/or other suitable material.

In some embodiments, the one or more frames of the present invention is constructed using a combination of structures and/or geometries (e.g., braided and zig-zag patterns). For example, in some embodiments, the frame may have a distal end having a braided configuration and a proximal end 140 having a wire frame with a zig-zag pattern along the cylindrical portion of the coil. In some embodiments, the frame comprises a middle portion (e.g., in between the distal and proximal ends) having a third structure/geometry that is the same, or different, than the proximal and/or distal ends. In some embodiments, the distal end, middle portion, and proximal end of the frame have the same structure/geometry. In some embodiments, the frame has a braided configuration.

In some embodiments, an implant (e.g., aortic root/arch implant) as disclosed herein comprises an expandable root/arch support structure.

In some embodiments, an expandable root/arch support structure comprises a central lumen of variable internal diameter. For example, in some embodiments, the expandable root/arch support structure comprises a first internal diameter at a proximal portion, a second internal diameter at a middle portion, and a third internal diameter at a distal portion of the expandable root/arch support structure, respectively. In some embodiments, the first internal diameter of the proximal portion, second internal diameter of the middle portion, and third internal diameter of the distal portion are the same. In some embodiments, the first internal diameter of the proximal portion, second internal diameter of the middle portion, and third internal diameter of the distal portion are different. In some embodiments, the first internal diameter of the proximal portion and third internal diameter of the distal portion are the same. In some embodiments, the second internal diameter of the middle portion is less than the first and third diameters of the proximal and distal portions, respectively.

In some embodiments, an expandable root/arch support structure comprises one or more openings having an internal diameter.

In some embodiments, the expandable root/arch support structure comprises one or more changes in a cross-sectional dimension within the body of the expandable root/arch support structure. For example, in some embodiments, a proximal end of the expandable root/arch support structure comprises a first cross-sectional dimension, a middle portion of the expandable root/arch support structure comprises a second cross-section dimension, and a distal end of the expandable root/arch support structure comprises a third cross-sectional dimension. In some embodiments, the first cross-sectional dimension is the same as the second and third cross-sectional dimensions. In some embodiments, however, the first cross-sectional dimension is different than the second and/or third cross-sectional dimensions. For example, in some embodiments, the first cross-sectional dimension and third cross-sectional dimension are greater than the second cross-sectional dimension.

In some embodiments, an expandable root/arch support structure has a coiled structure having a resting diameter.

In some embodiments, an expandable root/arch support structure has a coiled structure that is compressed to a diameter.

In some embodiments, an implant (e.g., aortic root/arch implant) as disclosed herein comprises one or more expandable branches comprising one or more distal expandable frames.

In some embodiments, an implant (e.g., aortic root/arch implant) as disclosed herein comprises one or more expandable branches comprising one or more telescoping frames.

In some embodiments, an implant (e.g., aortic root/arch implant) as disclosed herein comprises one or more expandable branches configured to pivot radially between 0 degrees and 360 degrees, relative to a first axis. In some embodiments, an implant (e.g., aortic root/arch implant) comprises one or more expandable branches configured to pivot laterally between 90 degrees and −90 degrees, relative to a second axis. In some embodiments, the first axis runs parallel to an elongate central passageway defined by the one or more expandable branches (e.g., a lumen). In some embodiments, the second axis runs perpendicular to the elongate central passageway defined by the one or more expandable branches (e.g., the second axis is parallel to the expandable root/arch support structure) (FIG. 9).

Other aspects of the disclosure relate to a prosthetic implant delivery system. In some embodiments, the prosthetic implant delivery system comprises an expandable root support structure comprising a first expandable branch and a second expandable branch. In some embodiments, the first expandable branch is placed over a first guide catheter, which is in turn, placed over a first balloon catheter. In some embodiments, the second expandable branch is placed over a second guide catheter, which is in turn, placed over a and a second balloon catheter. In some embodiments, a distal portion of the expandable root support structure is packaged within a distal sheath, the distal sheath being reversibly connected to a nosecone. In some embodiments, a proximal portion of the expandable root support structure comprises a valved conduit (e.g., porcine or bovine aortic valve). In some embodiments, an outer sheath extends from the proximal portion to the distal portion of the expandable root support structure. In some embodiments, prior to being deployed, the outer sheath of the prosthetic implant delivery system is in contact with the nosecone.

In some embodiments, the prosthetic implant delivery system comprises an expandable root support structure comprising a first expandable branch and a second expandable branch. In some embodiments, the first expandable branch is placed over a first guide catheter, which is in turn, placed over a first balloon catheter. In some embodiments, the second expandable branch is placed over a second guide catheter, which is in turn, placed over a and a second balloon catheter. In some embodiments, a distal portion of the expandable root support structure is packaged within a distal sheath, the distal sheath being reversibly connected to a nosecone. In some embodiments, the distal portion of the expandable root support structure comprises a valved conduit (e.g., porcine or bovine aortic valve). In some embodiments, an outer sheath extends from the distal portion to the proximal portion of the expandable root support structure. In some embodiments, prior to being deployed, the outer sheath of the prosthetic implant delivery system is in contact with the nosecone.

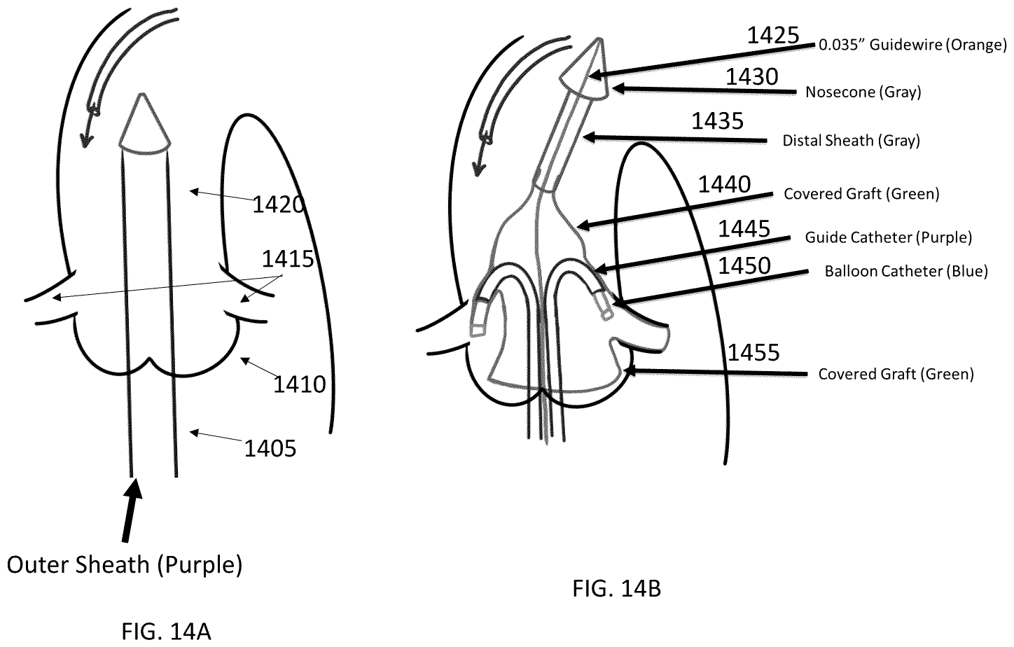

Other aspect of the disclosure relates to methods for treating a dissection, such as an aortic dissection. In some embodiments, the methods for treating the dissection use a transapical delivery approach. FIG. 14A shows delivery device 1405, housing an aortic root implant 1400, within an aortic root 1410 of a subject. In some embodiments, delivery device 1405 is advanced past the coronary ostia 1415 into an ascending aorta 1420 of a subject. FIG. 14B illustrates the various internal components of delivery device 1405, according to some embodiments. For example, in some embodiments, delivery device 1405 comprises guide wire 1425, nosecone 1430, distal sheath 1435, covered graft 1440, guide catheter 1445, balloon catheter 1450, and covered graft 1455.

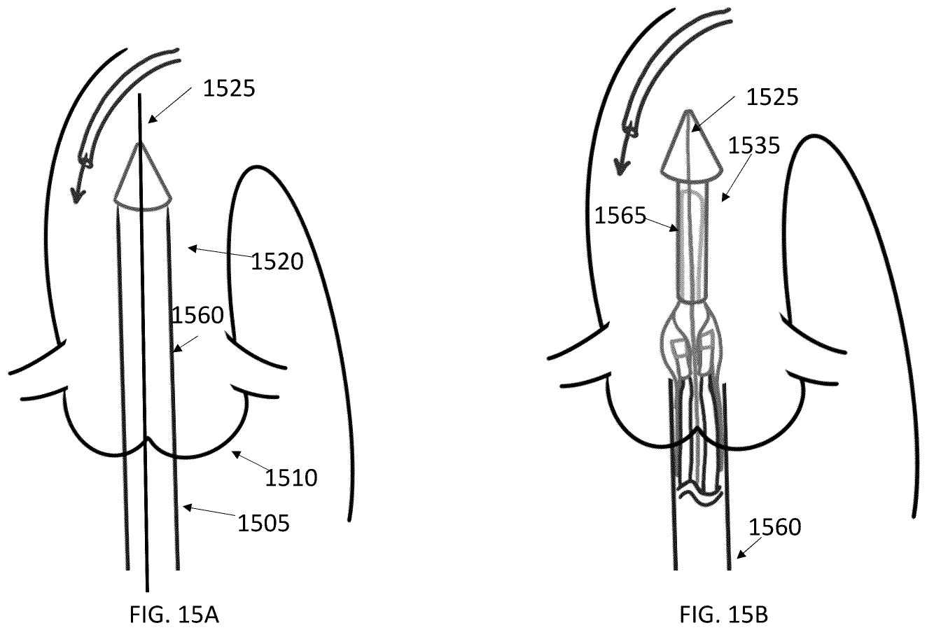

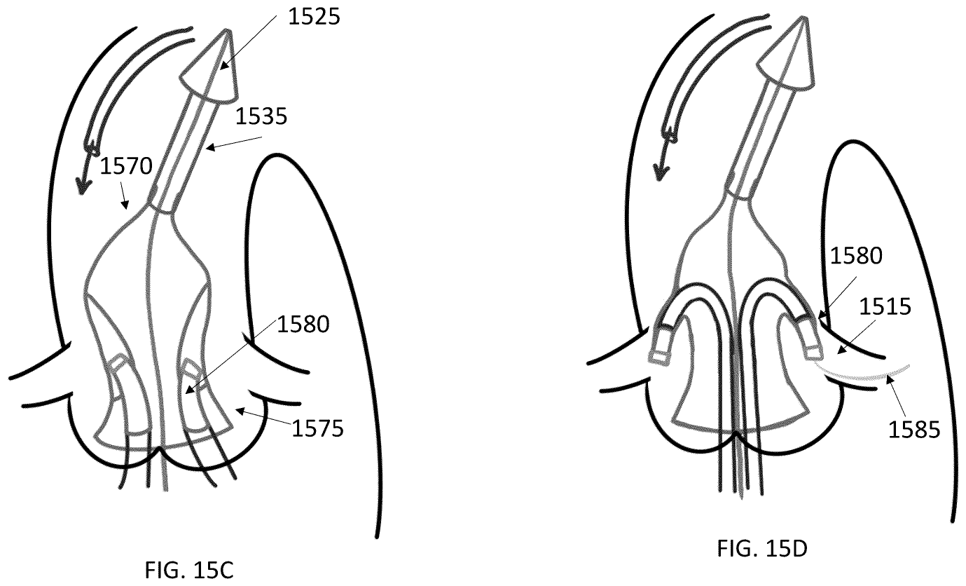

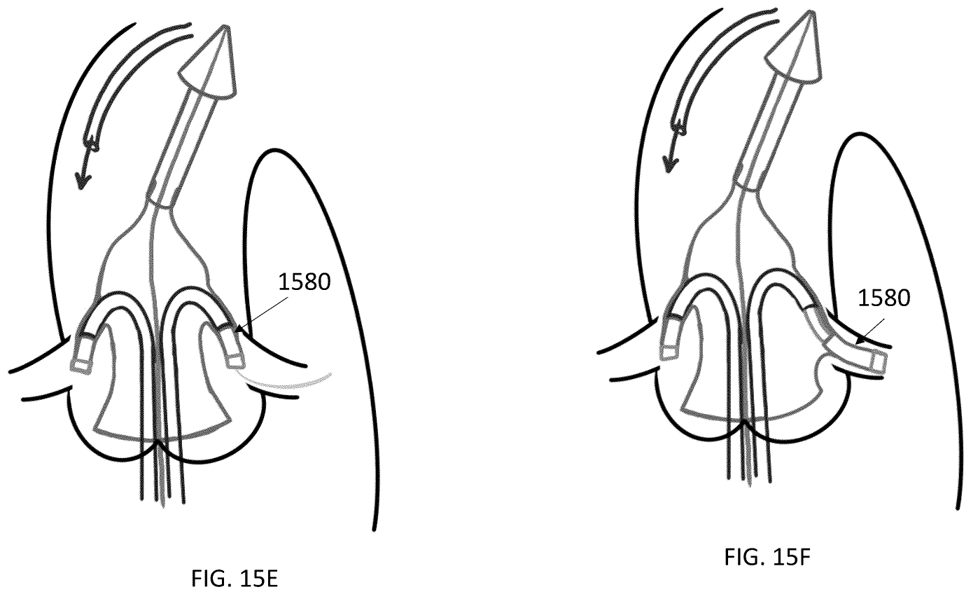

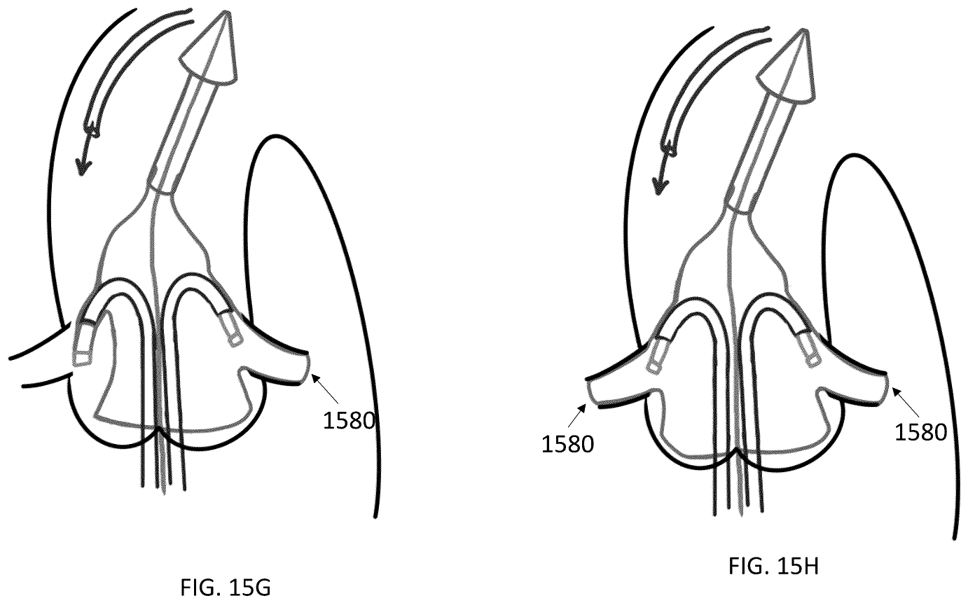

FIGS. 15A-15I illustrate placement of aortic root implant 1500 within aortic root 1510 of a subject using prosthetic implant delivery system 1505 via transapical delivery. In some embodiments, the methods comprise advancing a first guide wire 1525 into an ascending aorta 1520 of aortic root 1510. In some embodiments, the methods comprise advancing a prosthetic implant delivery system 1505 into the ascending aorta 1520 over the first guide wire 1525, the prosthetic implant delivery system 1505 comprising an outer sheath 1560 and a distal sheath 1535 extending through the outer sheath 1560, the distal sheath 1535 carrying a distal end 1565 of an expandable root support structure of aortic root implant 1500. In some embodiments, the methods comprise retracting the outer sheath 1560 in the ascending aorta 1520 to expose the distal sheath 1535 over the first guide wire 1525 (FIG. 15B). In some embodiments, the methods comprise advancing the distal sheath 1535 over the first guide wire 1525 to expose at least part of the distal portion 1570 of the expandable root support structure of aortic root implant 1500 (FIG. 15C). In some embodiments, the methods comprise retracting the outer sheath 1560 out of the ascending aorta 1520 to expose a proximal portion 1575 of the expandable root support structure, wherein the proximal portion comprises an aortic valve (FIG. 15C). In some embodiments, the methods comprise advancing a first expandable branch 1580 of the expandable root support structure into a coronary artery 1515 over a first coronary access wire 1585, wherein the first expandable branch 1580 comprises a telescoping structure comprising a proximal portion, a middle portion, and a distal portion, and wherein the first expandable branch 1580 is configured to pivot the between 0 degrees and 360 degrees, relative to a first axis (FIG. 15D-15H). In some embodiments, the methods comprise advancing the distal sheath 1535 to fully expose the distal portion 1570 of the expandable root support structure from the distal sheath 1535 (FIG. 15I).

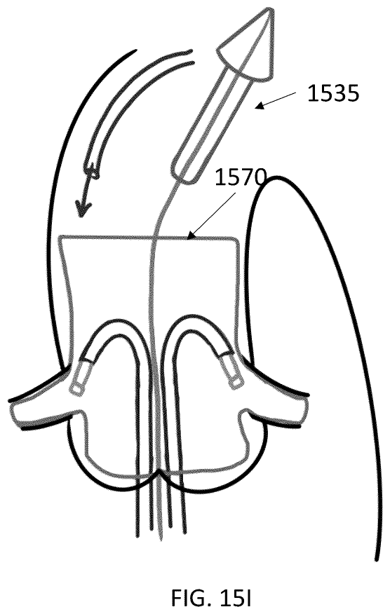

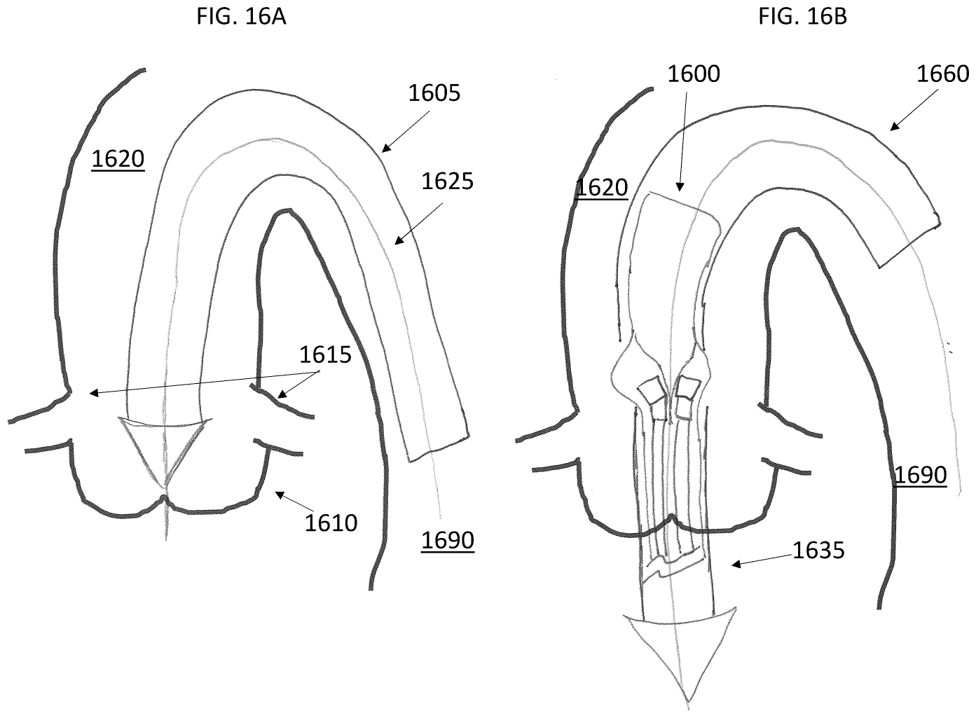

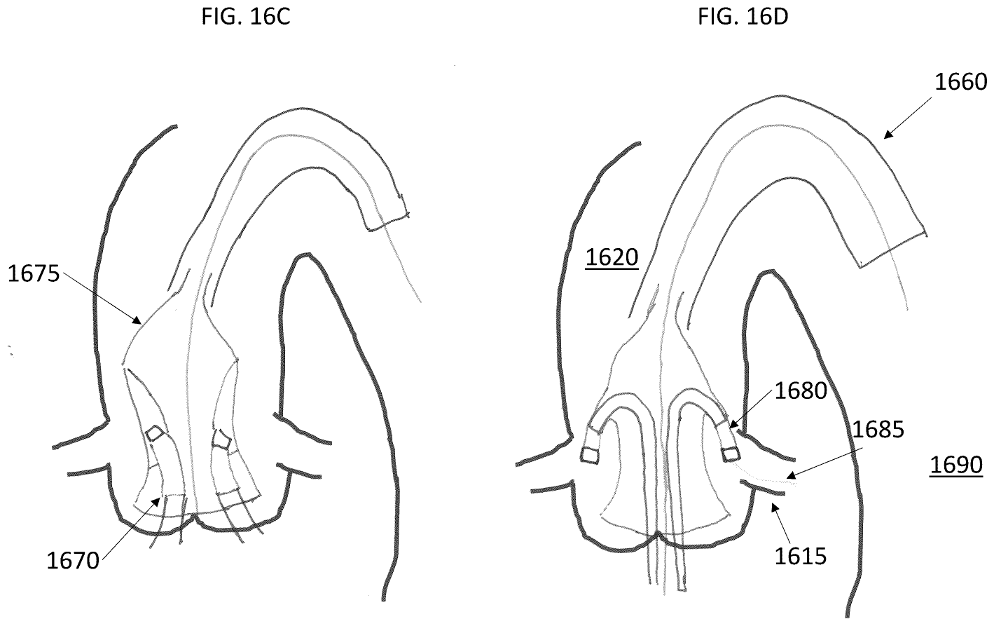

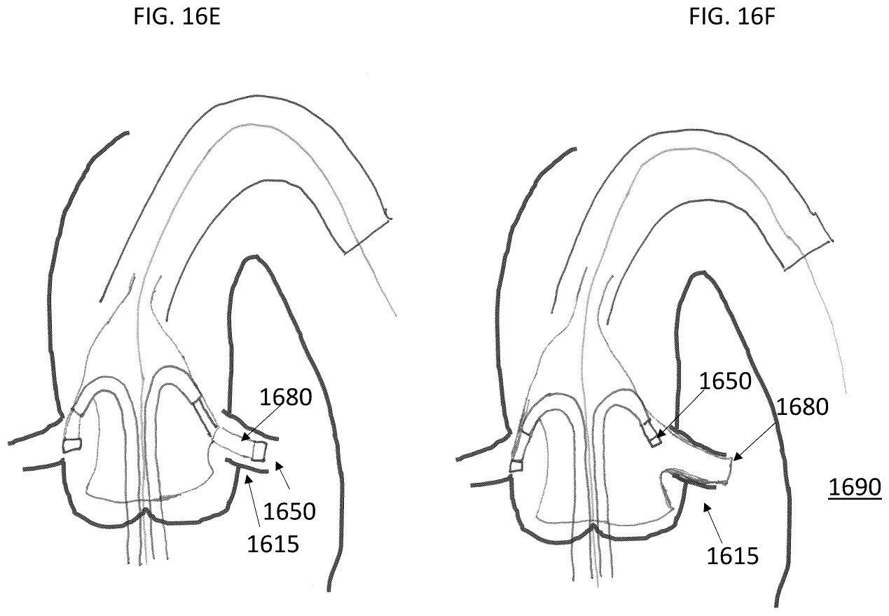

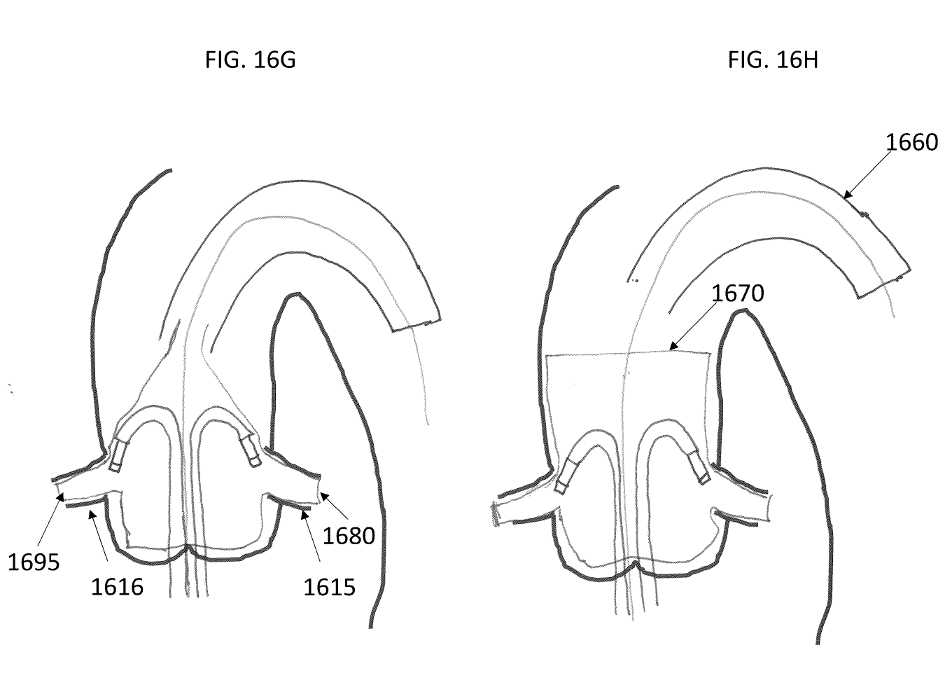

In some embodiments, the methods for treating the dissection use a transfemoral delivery approach. FIGS. 16A-16H illustrate placement of aortic root implant 1600 within aortic root 1610 of a subject using prosthetic implant delivery system 1605 via transapical delivery. In some embodiments, the methods of treating a dissection comprise advancing a first guide wire 1625 into a descending aorta 1690 and into an ascending aorta 1620. In some embodiments, the methods comprise advancing a prosthetic implant delivery system 1605 through the descending aorta 1690 and into the ascending aorta 1620 over the first guidewire 1625, the prosthetic implant delivery system 1605 comprising an outer sheath 1660 and a distal sheath 1635 extending through the outer sheath 1660, the outer sheath 1660 carrying an expandable root support structure of aortic root implant 1600. In some embodiments, the methods comprise retracting the outer sheath 1660 toward the descending aorta 1690 to expose the distal sheath 1635 over the first guidewire 1625 (FIG. 16B). In some embodiments, the methods comprise advancing the distal sheath 1635 out of the ascending aorta 1620 to expose a distal portion 1670 of the expandable root support structure, wherein the distal portion 1670 comprises an artificial valve (e.g., an aortic valve) (FIG. 16C). In some embodiments, the methods comprise, further retracting the outer sheath 1660 toward the descending aorta 1690 to expose at least a portion of a proximal portion 1675 of the expandable support structure. In some embodiments, the methods comprise advancing a first expandable branch 1680 of the expandable support structure into a first coronary artery 1615 over a first coronary access wire 1685, wherein the first expandable branch comprises a telescoping structure comprising a proximal portion, a middle portion, and a distal portion, and wherein the first expandable branch is configured to pivot the expandable branch between 0 degrees and 360 degrees, relative to a first axis. In some embodiments, the methods comprise retracting the outer sheath 1660 into the descending aorta 1690 to fully expose a distal portion 1670 of the expandable root support structure within the ascending aorta 1620 (FIG. 16H). In some embodiments, balloon catheter 1650 is used to expand the first expandable branch 1680 after placement within the first coronary artery 1615.

In some embodiments, the methods (e.g., transapical and/or transfemoral delivery further comprise advancing a second expandable branch, e.g., second expandable branch 1695 as shown in FIG. 16G, of the expandable root support structure into a second coronary artery 1616 over a second coronary access wire, wherein the second expandable branch 1695 comprises a telescoping structure comprising a proximal portion, a middle portion, and a distal portion, and wherein the second expandable branch is configured to pivot the between 0 degrees and 360 degrees, relative to a first axis.

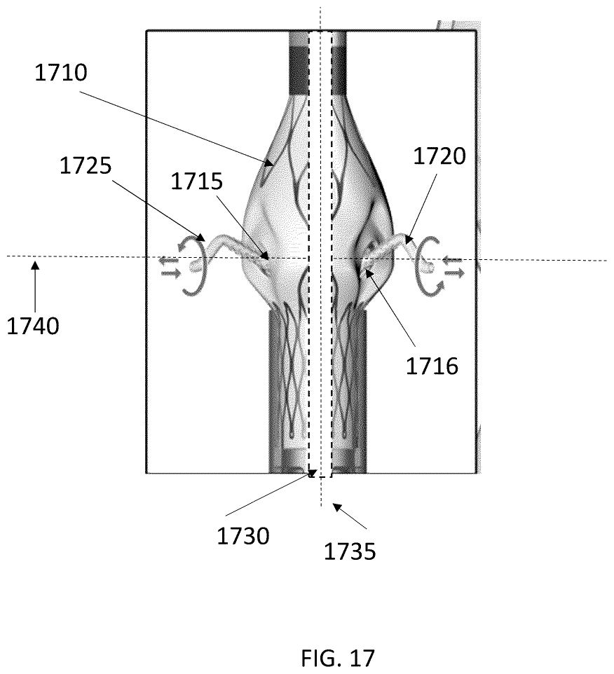

FIG. 17 provides an exemplary system for gaining coronary access using guide wires (e.g., as an alternative to using guide catheters and balloon catheters shown in FIGS. 14-16). FIG. 17 shows prosthetic implant delivery system 1705 with expandable root support structure 1710 of aortic root implant 1700 partially deployed. Expandable root support structure 1710 comprises first opening 1715 and second open 1716. In some embodiments, the gaining coronary access comprises maneuvering a first coronary access guide wire 1720 into first coronary artery, wherein after gaining coronary access, a second guidewire is passed through the first coronary access wire into the coronary artery. In some embodiments, the methods further comprise maneuvering a second coronary access guide wire 1725 into a second coronary artery, wherein after gaining coronary access, a third guidewire is passed through a second coronary access guide wire 1725 into the second coronary artery. First coronary access guide wire 1720 and second coronary access guide wire 1725, according to some embodiments, are configured to rotate about and move along an axis 1740 that is perpendicular to a longitudinal axis 1735 (e.g., that runs parallel to) a guide wire lumen 1730. (FIG. 17). In some embodiments, the coronary access wire is configured to rotate between 0 degrees and 360 degrees about an axis that is parallel to a line drawn between the two or more openings in the expandable root support structure.

In some embodiments, a telescoping structure of the first and/or second expandable branch conforms to the shape of the coronary artery.

In some embodiments, upon advancing a first expandable branch into a coronary artery, a distal portion of a first expandable branch is in a crimped state and one or more pores in the first expandable branch are open (e.g., patent), thereby allowing blood to pass through the graft during placement within the coronary artery.

In some embodiments, upon advancing a second expandable branch into a coronary artery, a distal portion of a second expandable branch is in a crimped state and one or more pores in the second expandable branch are open (e.g., patent), thereby allowing blood to pass through the graft during placement within the second coronary artery.

In some embodiments, the method further comprises inflating a balloon to expand the distal portion of the first and/or second expandable branch of the expandable arch support structure within the first and second coronary arteries, thereby anchoring the expandable root support structure to the native aorta. In some embodiments, expanding the distal portion of the first and/or second expandable branch closes one or more holes in the first and/or second expandable branch.

In some embodiments, a first coronary artery is the left coronary artery or the right coronary artery. In some embodiments, a second coronary artery is the left coronary artery or the right coronary artery.

In some embodiments, the prosthetic implant delivery system comprises a radiopaque marker (e.g., in the nosecone, guide catheter, or balloon catheter).

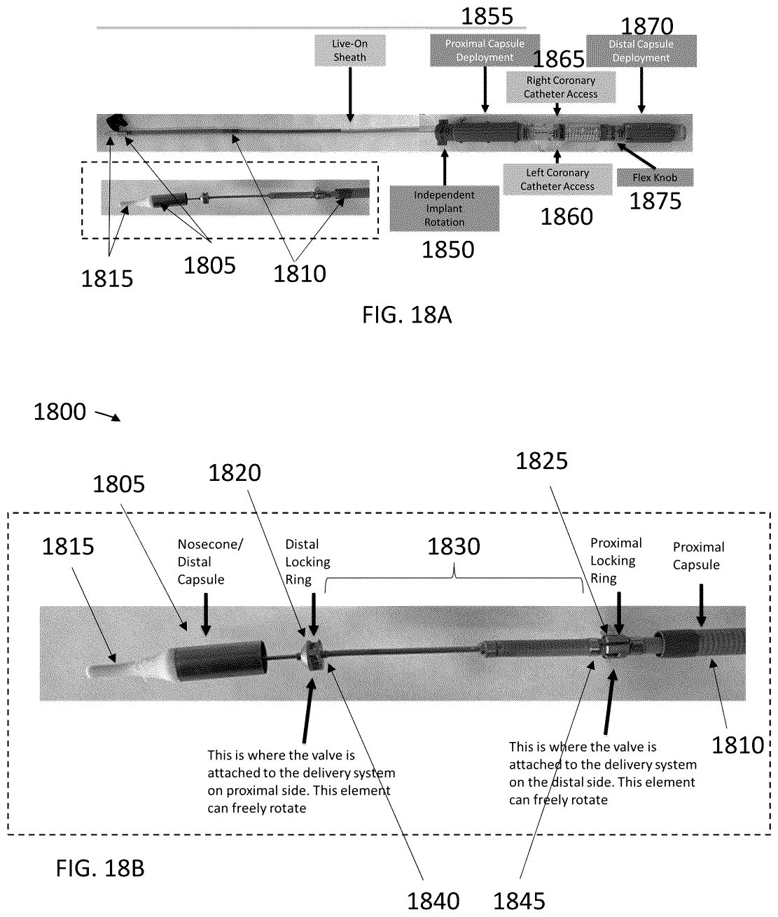

Other aspects of the disclosure relate to a graft delivery systems configured to enable rotation of the implant within the delivery device. FIGS. 18A shows an intact exemplary prosthetic implant delivery system 1800 comprising nosecone 1815, distal capsule 1805, proximal capsule 1810, independent implant rotation control 1850, proximal capsule deployment control 1855, left coronary catheter access control 1860, right coronary catheter access control 1865, distal capsule deployment control 1870, and flex knob 1875.

FIG. 18B shows an exemplary prosthetic implant delivery system 1800, wherein the distal capsule 1805 has been separated from the proximal capsule 1810 to reveal the internal components of the system. In some embodiments, the prosthetic implant delivery system 1800 comprises a distal capsule 1805 (e.g., comprising a nosecone 1815), a distal locking ring 1820, a proximal locking ring 1825, a proximal capsule 1810, and a prosthetic implant 1830. In some embodiments, the distal locking ring 1820 is configured to engage a distal end 1840 of the prosthetic implant 1830 (e.g., end comprising the artificial valve, useful for transfemoral delivery). Similarly, in some embodiments, the proximal locking ring 1825 is configured to engage a proximal end 1845 of the prosthetic implant (e.g., end comprising the artificial valve, useful for transapical delivery).

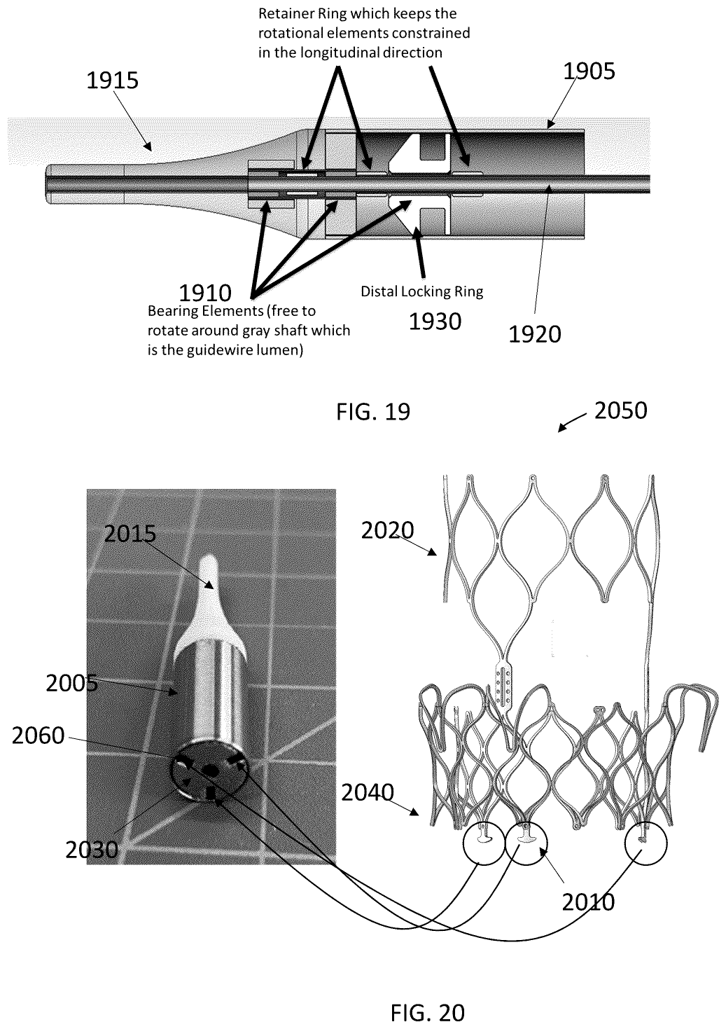

FIG. 19 shows a computer automated drawing illustrating an exemplary assembly of the nosecone and the distal locking ring shown in FIG. 18. As illustratively shown in FIG. 19, in some embodiments, the nosecone 1915 and distal capsule 1905 comprises a plurality of bearing elements 1910 that freely rotate around a guide wire lumen 1920. The nosecone 1915 and distal capsule 1905 further comprise a plurality of retainer rings 1925 that keep the rotational elements constrained in the longitudinal direction. Additionally, as shown illustratively in FIG. 19, the device also comprises a distal locking ring 1930 that is also flanked on both sides by a plurality of retainer rings 1925. The distal locking ring 1930 also freely rotates around the guide wire lumen 1920. While not shown explicitly in FIG. 19, a proximal ring may also be flanked on both sides by a plurality of retainer rings 1925 and can also freely rotate around the central elongate shaft.

The skilled artisan will understand that for transapical delivery methods, the proximal locking ring is configured to engage the portion of the expandable root support structure comprising an artificial valve. Likewise, for transfemoral delivery methods, the distal locking ring is configured to engage the portion of the expandable root support structure comprising the artificial valve.

In some embodiments a distal end of the expandable root support structure is operatively connected to the distal locking ring of the graft delivery system and a proximal end of the expandable root support structure is connected to the proximal locking ring. FIG. 20 illustrates an exemplary connection between the distal locking ring 2030 and a plurality of connectors 2010 at a distal end 2040 of inner frame 2020 of expandable root support structure 2050. As shown illustratively in FIG. 20, in some embodiments, the distal locking ring 2030 comprises a plurality of openings 2060. In some embodiments, the plurality of openings 2060 of the distal locking ring 2030 are configured to receive a plurality of connectors 2010 at a distal end 2040 of inner frame 2020 of expandable root support structure 2050. The connectors 2010 may have any suitable shape for attaching, mating, and/or being inserting into the openings 2060 of the distal locking ring 2030. Likewise, in some embodiments, a proximal locking ring comprises a second plurality of openings and a proximal end of the implant (e.g., expandable root support structure) comprises a second plurality of connectors. In some embodiments, the second plurality of openings of the proximal locking ring is configured to receive the second plurality of connectors of the proximal end of the implant. Other configurations are also possible.

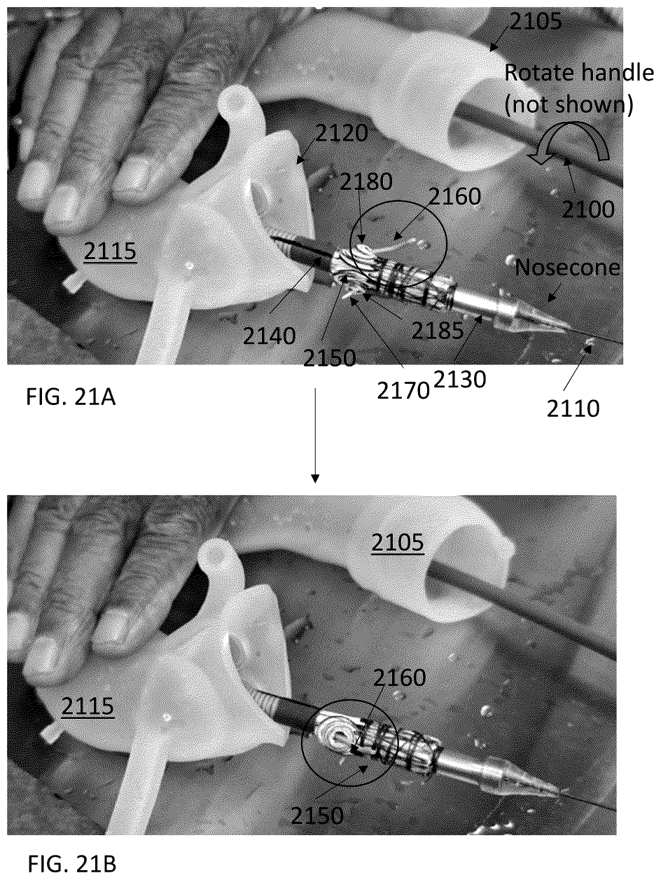

Those of skill in the art will understand that such configurations permit independent rotation of the prosthetic implant within the delivery system (e.g., the prosthetic implant can be rotated without rotating the delivery system). The inventors have discovered within the context herein that this rotatability may be useful for aligning an expandable branch of the expandable support structure with the coronary arteries. This independent rotation feature is shown illustratively in FIGS. 21A and 21B. FIG. 21A shows a prosthetic implant delivery system being administered transfemorally using a cast of a human aorta. As shown in FIG. 21, prosthetic implant delivery system 2100 is guided along a first guide wire 2110 up through descending aorta 2105 through the ascending aorta 2115 and out the aortic root 2120. Distal capsule 2130 is advanced out of the aortic root 2120 and proximal capsule 2140 is retracted toward the descending aorta 2105, thus exposing a portion of expandable root support structure 2150. In some embodiments, a first coronary catheter 2160 and a second coronary catheter 2170 pass through a first opening 2180 and a second opening 2185, respectively, in expandable root support structure 2150. In some embodiments, rotation of a handle (not shown in FIG. 21A) permits independent rotation of the loaded prosthetic implant (e.g., expandable root support structure 2150) without rotating the prosthetic implant delivery system 2100. This is clearly shown in FIG. 21B, wherein first coronary catheter 2160 has been rotated approximately 180 degrees.

The skilled artisan will understand that a prosthetic implant, and the delivery system, as disclosed herein may be used to treat any suitable disease or condition known to the skilled artisan. For example, in some embodiments, the implant (and/or delivery system) is configured to treat an aortic dissection. In some cases, the aortic dissection is a Type A aortic dissection. Treatment of other conditions is also contemplated herein, according to other embodiments. For example, in some embodiments, the implant (and/or delivery system) is configured to treat an intramural hematoma. Additionally, or alternatively, the implants (and/or delivery systems) disclosed herein may be used to treat a thoracic aortic aneurysm.

In some embodiments, a graft delivery system does not comprise rotatable elements (e.g., bearing elements, retainer rings, distal locking ring, or proximal locking ring). For example, in some embodiments, the graft delivery system comprises an outer sheath, the outer sheath configured to move along a first guidewire. In some embodiments, the system further comprises a distal sheath extending through the outer sheath, the outer sheath carrying an expandable root support structure. In some embodiments, the system comprises a first expandable branch of the expandable support structure inside the outer sheath, the first expandable branch configured to move along a first coronary access wire.

### Equivalents and Scope

While several embodiments of the present disclosure have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present disclosure. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present disclosure is/are used. Those skilled in the art will recognize or be able to ascertain using no more than routine experimentation, many equivalents to the specific embodiments of the disclosure described herein. It is, therefore, to be understood that the foregoing embodiments are presented by way of example only and that, within the scope of the appended claims and equivalents thereto, the disclosure may be practiced otherwise than as specifically described and claimed. The present disclosure is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present disclosure.

In cases where the present specification and a document incorporated by reference include conflicting and/or inconsistent disclosure, the present specification shall control. If two or more documents incorporated by reference include conflicting and/or inconsistent disclosure with respect to each other, then the document having the later effective date shall control.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.”

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

When the word “about” is used herein in reference to a number, it should be understood that still another embodiment of the disclosure includes that number not modified by the presence of the word “about.”

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

Any terms as used herein related to shape, orientation, alignment, and/or geometric relationship of or between, for example, one or more articles, structures, forces, fields, flows, directions/trajectories, and/or subcomponents thereof and/or combinations thereof and/or any other tangible or intangible elements not listed above amenable to characterization by such terms, unless otherwise defined or indicated, shall be understood to not require absolute conformance to a mathematical definition of such term, but, rather, shall be understood to indicate conformance to the mathematical definition of such term to the extent possible for the subject matter so characterized as would be understood by one skilled in the art most closely related to such subject matter. Examples of such terms related to shape, orientation, and/or geometric relationship include, but are not limited to terms descriptive of: shape-such as, round, square, circular/circle, rectangular/rectangle, triangular/triangle, cylindrical/cylinder, elliptical/ellipse, (n) polygonal/(n) polygon, etc.; angular orientation—such as perpendicular, orthogonal, parallel, vertical, horizontal, collinear, etc.; contour and/or trajectory—such as, plane/planar, coplanar, hemispherical, semi-hemispherical, line/linear, hyperbolic, parabolic, flat, curved, straight, arcuate, sinusoidal, tangent/tangential, etc.; direction—such as, north, south, east, west, etc.; surface and/or bulk material properties and/or spatial/temporal resolution and/or distribution—such as, smooth, reflective, transparent, clear, opaque, rigid, impermeable, uniform(ly), inert, non-wettable, insoluble, steady, invariant, constant, homogeneous, etc.; as well as many others that would be apparent to those skilled in the relevant arts. As one example, a fabricated article that would described herein as being “square” would not require such article to have faces or sides that are perfectly planar or linear and that intersect at angles of exactly 90 degrees (indeed, such an article can only exist as a mathematical abstraction), but rather, the shape of such article should be interpreted as approximating a “square,” as defined mathematically, to an extent typically achievable and achieved for the recited fabrication technique as would be understood by those skilled in the art or as specifically described. As another example, two or more fabricated articles that would described herein as being “aligned” would not require such articles to have faces or sides that are perfectly aligned (indeed, such an article can only exist as a mathematical abstraction), but rather, the arrangement of such articles should be interpreted as approximating “aligned,” as defined mathematically, to an extent typically achievable and achieved for the recited fabrication technique as would be understood by those skilled in the art or as specifically described.

What is claimed is:

1. A prosthetic implant, comprising:
an expandable root support structure comprising one or more openings and one or more expandable branches configured to be positioned within the one or more openings, and operably linked to the expandable root support structure, wherein the expandable root support structure is configured to be positioned within at least a portion of an ascending portion of a native aorta,
wherein the expandable root support structure comprises a valved conduit positioned at a proximal end of the expandable root support structure, and a non-porous layer that is configured to contact an outer wall of the native aorta,
wherein the one or more expandable branches comprises a telescoping structure comprising a proximal portion, a middle portion, and a distal portion, wherein an inner diameter of the proximal portion is larger than an inner diameter of the distal portion,
wherein the one or more expandable branches is configured to be positioned within at least a portion of at least one coronary artery of an aortic root and to permit blood flow from within the expandable root support structure and into at least one coronary artery of the aortic root.

2-5. (canceled)

6. The prosthetic implant as in claim 1, wherein the proximal portion of the one or more expandable branches comprises a telescoping frame.

7-20. (canceled)

21. The prosthetic implant as in claim 1, wherein the one or more expandable branches further comprise a middle portion comprising a telescoping metallic wire frame.

22-23. (canceled)

24. The prosthetic implant as in claim 1, wherein the distal portion of the one or more expandable branches comprises a distal expandable frame.

25-28. (canceled)

29. The prosthetic implant as in claim 24, wherein, when the distal expandable frame of the distal portion is in a crimped state and the middle portion is in a collapsed state, the distal and middle portions are configured to fit within the proximal portion and/or within a lumen of the expandable root support structure.

30. The prosthetic implant as in claim 1, wherein the proximal portion of the one or more expandable branches is operably linked to the middle portion of the one or more expandable branches and the middle portion of the one or more expandable branches is operably linked to the distal portion of the one or more expandable branches.

31. The prosthetic implant as in claim 1, wherein the one or more expandable branches is configured to pivot radially between 0 degrees and 360 degrees, relative to a first axis, and to pivot laterally between 90 degrees and −90 degrees, relative to a second axis.

32. (canceled)

33. The prosthetic implant as in claim 1, wherein the one or more expandable branches is configured to be in either an extended state or in a collapsed state.

34-43. (canceled)

44. The prosthetic implant as in claim 1, wherein the expandable root support structure comprises a first expandable branch configured to be positioned within at least a portion of a left coronary artery, and a second expandable branch is configured to be positioned within at least a portion of a right coronary artery.

45-46. (canceled)

47. The prosthetic aortic implant as in claim 1, wherein the distal portion of a first expandable branch and the distal portion of a second expandable branch each comprise a distal expandable frame, that when expanded, is configured to apply a radially outward force to a coronary artery, thereby anchoring the implant to the native aorta.

48-50. (canceled)

51. The prosthetic implant as in claim 1, wherein the proximal end of the expandable root support structure is in contact with but not directly adhered and/or grafted to the native aorta upon deployment in the native aorta.

52-71. (canceled)

72. The prosthetic implant as in claim 1, wherein any one of the expandable root, telescoping frames, and/or distal expandable frames further comprises a non-porous layer.

73. The prosthetic implant as in claim 1, wherein the non-porous layer is configured to expand and, upon expansion, apply a radially outward force to the ascending aorta thereby forming a seal against an internal surface of the aorta and/or anchoring the prosthetic implant to the native aorta.

74. The prosthetic implant as in claim 1, wherein the non-porous layer is configured to expand and, upon expansion, apply a radially outward force to at least one coronary artery thereby forming a seal against an internal surface of the coronary artery and/or anchoring the prosthetic implant to the native aorta.

75. (canceled)

76. The prosthetic implant as in claim 1, wherein the non-porous layer expands due to a blood hydrostatic pressure created by blood flowing through an intraluminal space formed between the non-porous layer and a porous layer.

77-78. (canceled)

79. The prosthetic implant as in claim 1, wherein the non-porous layer is positioned across, and configured to seal around, at least a portion of a dissection.

80-87. (canceled)

88. The prosthetic implant as in claim 1, wherein the expandable root support structure, configured to be positioned within an ascending portion of the native aorta, is coupled to an expandable arch support structure configured to be positioned within at least a portion of a descending portion of the native aorta.

89. The prosthetic implant as in claim 1, wherein the implant is configured to treat an aortic dissection.

90-93. (canceled)

94. A method of treating a dissection comprising:
advancing a first guide wire into a descending aorta into an ascending aorta;
advancing a prosthetic implant delivery system through the descending aorta and into the ascending aorta over the first guide wire, the prosthetic implant delivery system comprising an outer sheath and a proximal sheath extending through the outer sheath, the outer sheath carrying an expandable root support structure;
retracting the outer sheath toward the descending aorta to expose the proximal sheath over the first guide wire;
advancing the proximal sheath out of the ascending aorta to expose a proximal portion of the expandable root support structure, wherein the proximal portion comprises an aortic valve;
further retracting the outer sheath toward the descending aorta to expose at least a portion of a distal portion of the expandable support structure,
advancing a first expandable branch of the expandable support structure into a coronary artery over a first coronary access wire, wherein the first expandable branch comprises a telescoping structure comprising a proximal portion, a middle portion, and a distal portion, and wherein the first expandable branch is configured to pivot the expandable branch between 0 degrees and 360 degrees, relative to a first axis, and
retracting the outer sheath into the descending aorta to fully expose distal portion of the expandable root support structure within the ascending aorta.

95. A graft delivery system, the system comprising:
a guide wire lumen;
a distal capsule comprising a plurality of bearing elements;
a distal locking ring and a proximal locking ring; and
a prosthetic implant system comprising an expandable root support structure,
wherein a proximal end of the expandable root support structure is connected to the proximal locking ring,
wherein a distal end of the expandable root support structure is connected to the distal locking ring, and
wherein the plurality of bearing elements, the distal locking ring, and the proximal locking ring are configured to rotate about a guide wire lumen.

96-110. (canceled)
